(19) United States
(12) Patent Application Publication
(10) Pub. No.: US 2008/0000474 A1
(43) Pub. Date: Jan. 3, 2008

(54) Apparatus for Administering a Breathable Gas, and Components Thereof

(75) Inventors: Knut Jochle, Schondorf (DE); Dieter Heidmann, Castle Hill (AU); Rainer Jakobs, Munchen (DE); Dieter Klaus, Maulburg (DE); Adel Nibu, Munich (DE); Johann S. Burz, Wielenbach/Wildzhofen (DE); Bernd Lang, Grafelfing (DE); Stefan Madaus, Grafelfing (DE); Harald Vogele, Gauting (DE)

Correspondence Address: NIXON & VANDERHYE, PC, 901 NORTH GLEBE ROAD, 11TH FLOOR, ARLINGTON, VA, 22203, US

(73) Assignee: Map Medizin-Technologie GmbH, Martinsried (DE)

(21) Appl. No.: 11/666,350

(22) PCT Filed: Oct. 26, 2005
(86) PCT No.: PCT/EP05/11467

§ 371 Date: May 23, 2007

(30) Foreign Application Priority Data
Oct. 26, 2004 (DE) 102004052054.2
Nov. 4, 2004 (DE) 102004053324.5
Jan. 5, 2005 (DE) 102005000819.4

(51) Int. Cl. A61M 16/00; A61M 16/20; A62B 7/00

(52) U.S. Cl. 128/204.18; 128/204.21; 128/204.22; 128/205.23

(57) ABSTRACT

A modular insert is provided for defining an air path for use with CPAP, VPAP, auto CPAP or a ventilator. The modular air path insert may be provided with one or more sound suppressing structures, e.g., to act as mufflers. The insert can be readily exchanged or replaced, e.g., for hygienic purposes.

### CROSS-REFERENCES TO PRIORITY APPLICATIONS

This application claims the benefit of German Application No. 102004052054.2 filed Oct. 26, 2004, German Application No. 102004053324.5 filed Nov. 4, 2004, and German Application No. 102005000819.4, filed Jan. 5, 2005, each incorporated herein by reference in its entirety.

### BACKGROUND OF THE INVENTION

The invention relates to an apparatus for administering a breathable gas, in particular room air, at a pressure level that is at least in some phases above the ambient pressure. The invention also relates to components of such an apparatus. In particular, the invention is directed to a device, in particular a Continuous Positive Airway Pressure (CPAP) device, for treatment and/or diagnosis of sleep-related breathing problems by bringing about a pneumatic splinting effect in the region of the upper airways.

In CPAP devices, the breathing gas delivered to a user is typically pumped by a pumping device to a pressure level that is above the ambient pressure. This pumping device can in particular be embodied as a blower, with a motor-driven impeller in the form of an axial, semiaxial, or radial impeller. The pumping device per se forms a pressure lock, by means of which a portion of an air guidance system toward the patient is at a higher pressure than an intake-side portion of the air guidance system that is open to the environment. Conducting the breathing gas on from this pumping device to a user can be done via a flexible hose and a breathing mask device connected to it. Typically a patient uses these devices at home, where they can be placed in the immediate vicinity of the patient's pillow, for instance on a night table.

Thus to attain the greatest possible treatment comfort, CPAP devices are typically embodied such that they cause the least possible operating noise. To reduce the operating noise, it is possible in particular to provide sound-absorbing air guidance structures in the interior of the CPAP device, for absorbing any acoustic events that are coupled into the breathing gas path by the pumping device. These air guidance structures may in particular be lined with a sound-absorbing material. These sound-absorbing materials are typically foam material or material that otherwise has pores, such as foam. Cleaning this air guide proves relatively problematic.

One such apparatus is known particularly from the present Applicants' International Patent Application WO 01/10489 incorporated herein by reference in its entirety. This apparatus includes a basic unit and an air humidifier device that can be coupled in modular fashion to it. The basic unit in turn includes a housing device and a blower device located in the housing device. The blower device is incorporated into a breathing gas path, which on both the suction side and the pressure side is embodied as a labyrinth structure, for absorbing any running noises that the blower device may have.

Depending on the configuration of a CPAP device, a patient's exhaled breathing gases may get back into the CPAP device. The level of patient's exhaled breathing gases getting back into the CPAP device is influenced by factors such as the length of the breathing gas hose; the breathing gas rinsing flow toward the mask; and the patient's inhalation volume. Cleaning these CPAP devices is typically performed by skilled persons skilled in the equipment and furthermore proves to be relatively time-consuming.

To overcome the problem of cleaning the air path, disposable or sterilisable air paths were designed. German patent DE 2941554 discloses a sterilisable air cartridge that can be used in medical devices such as ultrasound atomizers. Wickham et al have three US patents (U.S. Pat. Nos. 6,302,105; 6,629,528; and 6,899,100, each incorporated by reference in its entirety) relating to a sterilisable or disposable air path. However, these devices are not designed to allow inexpensive manufacture of a range of devices that deliver a range of different therapy modes.

### BRIEF SUMMARY OF THE INVENTION

One aspect of the invention is to create an apparatus of the type defined at the outset that is distinguished by highly convenient manipulation and also proves advantageous from the standpoint of manufacture, assembly, and maintenance, including hygienic and noise aspects.

One embodiment of the invention relates to an apparatus for administering a breathable gas, in particular at a pressure level that at least in some phases is above the ambient pressure, having a delivery device for delivering the breathing gas via a breathing gas delivery path, a breathing gas inlet region, located upstream of the delivery and forming part of the breathing gas delivery path, and an outlet region, downstream of the delivery device and likewise forming part of the breathing gas delivery path. The breathing gas inlet region and the breathing gas outlet region are furnished by a first module having at least one sound-absorbing structure.

Another embodiment of the invention relates to an apparatus for administering a breathable gas, in particular at a pressure level that at least in some phases is above the ambient pressure, having a delivery device for delivering the breathing gas via a breathing gas delivery path, a breathing gas inlet region, located upstream of the delivery device and forming part of the breathing gas delivery path, an outlet region, downstream of the delivery device and likewise forming part of the breathing gas delivery path, and a housing structure. The apparatus in its construction is broken down into a first module and a second module, and the first module includes the delivery-path components that serve to furnish the breathing gas delivery path, and the second module includes the apparatus components that can be separated from the delivery path components.

Yet another embodiment of the invention relates to a device for delivering a breathing gas, having a air delivery device for pumping the breathing gas to a pressure level that is above the ambient pressure, and having a basic unit for receiving the air delivery device, in which the air delivery device forms part of a first module, and the basic unit forms a second module, which is divided up into a lower structural part and an upper structural part, and the two structural parts can be put into an open position toward one another, and in this open position the first module can be removed from or introduced into the second module.

Aspects of the invention may make it possible to break the equipment down into one group that requires regular maintenance or cleaning and a second group that requires less cleaning and maintenance. Aspects of the invention make it possible to replace the structures that enter into communication with the breathing gas in a replacement operation, for example, in a way similar to how a toner cartridge is handled for a laser printer, whilst the electronic components may continue to be used. Furthermore, embodiments of the invention make it possible to construct various equipment configurations inexpensively using modules that are compatible both geometrically and in terms of circuitry.

The first module preferably includes the breathing gas line portion that defines the breathing gas inlet region; this breathing gas line portion that defines the breathing gas inlet region is preferably provided with a sound damping unit.

The first module preferably includes a breathing gas line portion that defines the breathing gas outlet region. This breathing gas line portion that defines the breathing gas outlet region is likewise preferably provided with a sound damping unit.

According to a preferred embodiment of the invention, the first module includes a first module housing, which is embodied as a thin-walled structure. This thin-walled structure may be made from a plastic material or preferably from a foil- or film-coated cardboard material. The first module housing receives the sound damping units and the delivery device and may be a disposable item. It is possible to design the first module in terms of its construction such that the first module or the first module housing is reinforced by structures enclosed in the first module housing, in particular the aforementioned sound-absorbing structures. This makes it possible to prevent swelling of the first module housing in response to the breathing gas pressure that may prevail downstream of the delivery device.

In an especially preferred embodiment of the invention, the first module housing is designed to include defined breathing gas duct portions, in particular an intake portion and an outlet portion. These breathing gas duct portions may be exposable by removing cover portions. These cover portions may be formed directly by the wall material of the first module. The cover portions to be removed may be defined by perforated places in the first module housing.

In the first module housing, it is possible to provide a connection portion for connecting the delivery device. The delivery device may either be attached to the first module or, in an especially preferred embodiment, be inserted at least partway into the first module. Particularly in the latter case, the delivery device may be received in a receiving portion in the first module. This receiving portion may be embodied as a recess, for receiving an insert that includes the delivery device.

It is possible to design the first module such that the delivery device is initially integrated with the first module and upon replacement of the first module is removable from the first module. The removal of the delivery device from the first module is preferably accomplished with relatively simple disassembly and preferably largely without tools. As a result, it becomes possible to process the delivery device for a further use.

The first module is preferably designed such that measuring instruments, or structures such as tubular stubs intended for forming breathing gas line portions can be coupled to it. The docking may be accomplished in conjunction with fasteners that are already fixed in advance to the first module housing.

The delivery device is preferably embodied as a blower. The blower device includes a motor having an impeller coupled thereto. The impeller is preferably a radial wheel with backward-curved vanes. It is possible to embody the blower device as a disposable component. However, it is also possible to construct the blower device in parts in such a way that the components that are relevant to sterilization and cleaning can be disconnected from the actual impeller drive device. The kinematic coupling of the impeller device to the drive device can be accomplished in particular by means of a magnet coupling. However, it is also possible to embody the impeller such that it functions as part of the motor, in particular as the motor rotor, for instance a short-circuit rotor.

The first module may be constructed so that it is subdivided into a plurality of component elements. For instance, it is possible for the portion of the breathing gas delivery path on the intake side and the portion of the breathing gas delivery path on the compression side to be embodied as separate disposable modules. The delivery device for delivering the breathing gas may also be integrated with a basic unit provided for receiving the first module.

The sound damping unit provided in the region of the breathing gas inlet region is preferably integrated, as a replaceable inlet sound damper unit, with the first module. The sound damping unit provided in the region of the breathing gas outlet region is preferably also integrated, as a replaceable outlet sound damper unit, with the first module. It is possible to combine the two sound damper units into an interchangeable unit, which can be changed in the course of a maintenance or sterilization operation.

Furthermore, the first module preferably includes a transducer, for picking up a measurement signal that is indicative of the flow of breathing gas. This transducer may be embodied as a differential pressure transducer. The differential pressure required for raising this measurement signal can be built up intrinsically via the breathing gas path. It is also possible to embed structures suitable for building up a differential pressure in the breathing gas path. It is also possible in the region of the breathing gas path to provide other kinds of sensor structures, by way of which a signal indicative of the breathing gas flow can be raised.

Preferably, by means of the first module housing, connection devices are furnished for coupling further elements of the region of the breathing gas delivery path located downstream of the delivery device, in particular for direct coupling of a breathing gas hose. The connection devices of the first module are preferably embodied and disposed such that they are accessible via a connection portion that is bounded by the second module.

It is advantageously possible to embody the first module and the second module as components substantially stacked one above the other. The second module may in particular be embodied such that it includes an upper housing device; the equipment operating devices and in particular the equipment electronics are integrated with the second module.

The second module preferably also includes a mains voltage connection structure. The mains voltage connection structure may furthermore be provided with a mains switch, provided for manual actuation, and this mains switch is disposed in the immediate vicinity of the mains voltage connection structure or of where a power cord enters the second module. The connection of the power cord to the second module can be made via an outlet, and the mains switch may be combined with the outlet to make a structural unit. The second module may also be coupled with a power pack.

In a preferred embodiment of the invention, the second module includes a lower structural part intended for receiving the first module and an upper structural part covering this lower structural part, forming an upper equipment face.

In a further preferred embodiment of the invention, the upper structural part is pivotably coupled to the lower structural part. This type of coupling can be attained in particular using pivot pins or pivot guide portions, which are embodied correspondingly complementary to the two structural parts.

In an especially preferred embodiment of the invention, both the upper structural part and the lower structural part each form one housing half-shell. These housing half-shells are preferably embodied such that after the upper structural part has pivoted upward, the first module can be inserted from above into the lower structural part. It is possible to embody the coupling of the two structural parts, in particular the joint structure serving the purpose of the pivotable coupling, such that the structural parts are separable from one another in a defined pivoted position, so that in particular the upper structural part can be removed from the lower structural part.

It is also possible to embody the joint structure, preferably intended for coupling the two structural parts, such that the pivot axis defined by the joint structure extends in the region of the front or optionally also rear crosswise edge of the basic unit. If the joint structure is embodied such that this pivot axis extends in the region of the front crosswise edge of the basic unit, then it becomes possible to lift the upper structural part in the rear portion of the basic unit and pivot it forward. In this pivoted state of the basic unit, the first module can be inserted into the lower structural part from behind, without requiring that the upper structural part be removed completely from the lower structural part for the purpose.

In an especially preferred embodiment of the invention, a control circuit and a voltage supply circuit are detachably disposed in the region of the upper structural part. This control circuit and the voltage supply circuit are preferably divided into separate electrical modules. In particular, it is possible to embody these electrical modules such that certain user-control-relevant portions of these modules are open to the environment through recesses or openings embodied in the upper structural part and are thus accessible to a user. Moreover, it becomes possible to embody the electrical modules such that voltage supply circuits or switches provided in them are also freely accessible at the rear of the device. It is also possible to embody the electrical modules such that any air throughput opening portions required for cooling the electrical components of these modules are freely accessible in the rear region of the device of the invention and/or toward the top of the device.

The basic unit formed by the first and second modules is preferably designed such that the equipment furnishes a connection device for selective connection of an air humidifier. In the region of this connection device the basic unit may be designed such that after docking of an air humidifier, the result is a largely closed structure. A covering element may be provided to cover the region surrounding the connection device when the equipment is used in a configuration without an air humidifier attached, thus maintaining an attractive external appearance of the basic unit in either configuration with or without the air humidifier.

In a preferred embodiment of the invention, line portions are furnished in the region of the upper structural part, for creating an electrical connection between the electrical modules, inserted into the upper structural part, and optionally to the first module and/or also to the air humidifier device. This line system (or common rail system) can be embodied such that the corresponding electrical docking of the electrical modules, of the first module, and/or of the air humidifier device is achieved when these modules are inserted properly into the upper structural part. Alternatively, or in combination with this provision, it is also possible to embody the aforementioned electrical modules such that any required electrical connections between these modules are attained when these electrical modules are disposed suitably side by side in the upper structural part or are placed onto the first module. It is possible for the modules to be embodied such that control-relevant signals are communicated in wireless fashion, in particular optically or in some other way electromagnetically or field-electrically. For instance, it is possible to perform a voltage supply for a heater of the air humidifier via the power pack, and the power pack has a signal input interface for receiving a signal intended for triggering a power switch. This signal can be furnished via the control module.

The air humidifier preferably includes a cup portion and a cap element closing off the cup portion. The cap element preferably defines an air supply path, for creating a connecting path between the outlet portion of the first module and the inner region of the cup portion. The cap element may be embodied such that it includes a connection stub for connecting a breathing gas line. This connection stub is preferably embodied integrally with the cap element.

In a particularly preferred embodiment of the invention, the air humidifier is provided with an electric heater. The heater may be formed by a heating bar. The heating bar is preferably incorporated detachably into the air humidifier. It is possible to incorporate the heating bar into the air humidifier of the cap element, in particular by providing that the heating bar is detachably coupled with the cap element.

It is possible to embody the air humidifier such that the heating bar is guided all the way through the cap element in a sealing manner. The heating bar may be detachably locked to the cap element, and may be provided directly with an electrical connection device embodied on an end portion of the heating bar. The electrical connection device may be embodied such that in the course of coupling the air humidifier to the basic unit, it likewise couples with corresponding electrical connection elements of the basic unit.

The cap device preferably includes a partition element, for separating an airway extending in the cap element, from some portions of the inner region of the cup portion. This partition element is preferably removable, so that the airway extending in the cap element is advantageously freely accessible for cleaning purposes. The partition element may be made from an elastomer material and may at the same time perform sealing tasks for sealing off the cup portion and/or the heating bar element.

It is possible to provide a storage device, for instance in the form of a memory chip, in a component associated with a blower device, in particular in a motor plug, by way of which blower device or chip hygiene-relevant information or operation-relevant information and data can be recorded.

It is possible to equip the hygiene-relevant components with a display, such as thermal paper labels, that make it possible to ascertain whether or to what extent the applicable component has already been used up. The change in color of the thermal paper label can be brought about using small heating resistors located in the vicinity of the display. Via such a display, it is also possible to implement a display of hours in operation.

It is also possible to detect other kinds of hygiene-relevant events and to record information accordingly. For instance, the hygienic condition can be ascertained as a function of the rebreathing volume. The corresponding information may be obtained from a breath flow signal that is raised, for instance, via a differential pressure sensor.

As a result, it advantageously becomes possible to create a breathing gas delivery device, in particular a CPAP device, in which an air feeding module that is relevant to hygiene can be removed from or inserted into the applicable receiving system in a way similar to how a toner cartridge is handled for a laser printer.

One device of the invention is preferably embodied such that the hygiene-relevant first module is replaceably insertable into the lower structural part. The electrical modules required for performing the control operations as well as the voltage supply are preferably detachably inserted into the upper structural part. The concluding electrical and mechanical coupling of these modules is preferably done whenever the upper structural part, equipped with the electrical modules, is pivoted over or otherwise moved onto the lower structural part. It is possible to embody the electrical modules such that an intrinsically functional breathing gas supply device can be constructed by simply placing these electrical modules against the first module, and in that case the hinged housing system can initially be dispensed with. In an alternative embodiment, it becomes possible to construct an abbreviated system suitable for a patient from the hygiene-relevant first module and the electrical modules and to perform an examination of the patient initially only with this abbreviated configuration. If a need for therapy for the examined patient is found, then the system used for the examination can be supplemented with the upper and lower structural parts of the second module and thus assembled into a therapy device that is both aesthetically attractive and suitable for use at home.

The two structural parts are preferably made from a plastic material. The electrical modules are preferably embodied such that different device variants can be made by suitable selection and combination of various compatible individual modules. The control circuit, or the module carrying this control circuit, may be part of a module family that furnishes control circuits by which modules are furnished that differ in terms of the complexity of control or in terms of the required control characteristics. It is also possible to embody the control circuit or the control module such that the appropriate configuration of this module in terms of regulation can be software-based, in particular by inserting a memory card element that specifies the pressure regulating concept.

### BRIEF DESCRIPTION OF THE DRAWINGS

Further details and characteristics of the invention will become apparent from the ensuing description in conjunction with the drawings.

FIG. 1 is shows a perspective view of a disposable first module according to an embodiment of the invention having perforated opening zones;

FIG. 2 shows a simplified sectional view of one embodiment of the internal construction of the first module of FIG. 1;

FIG. 3 shows a perspective sketch of a first module according to an embodiment of the invention with an integrated delivery device;

FIG. 4 shows a sketch of the first module of FIG. 3 with an opened top and with the delivery device removed;

FIG. 5 shows a sketch of the breathing gas line portions in a first module according to an embodiment of the present invention;

FIG. 6 shows a sketch of a first module according to an embodiment of the invention received in the interior of a second module;

FIG. 7 shows a sketch of a first module with a receiving portion for receiving a delivery device according to an embodiment of the present invention;

FIG. 8 shows a sketch of a receiving portion for a delivery device embodied in the first module;

FIG. 9 shows a sectional view of an air humidifier device that can be attached to a first module of an embodiment of the invention;

FIG. 10 shows a simplified perspective view of an attachment part provided for furnishing a breathing gas stub according to an embodiment of the present invention;

FIG. 11 shows a perspective view of the air humidifier device of FIG. 9 on a first module of an embodiment of the invention;

FIG. 12 shows a sketch of a further embodiment of the invention for furnishing a breathing gas line device coupled in a sealing manner to the first module;

FIG. 13 shows a perspective sketch of a further embodiment of the invention of a connection region of a first module of an embodiment of the invention;

FIG. 14 shows a sketch of a further concept for furnishing a stub portion on the first module of an embodiment of the invention;

FIG. 15 shows a sketch of a concept according to the present invention for picking up a differential pressure signal, indicative of the breathing gas flow, using two pressure-measurement line portions that discharge into different chambers of the first module.

FIG. 16 is a perspective view of an apparatus according to a further embodiment of the invention with an air humidifier coupled thereto;

FIG. 17a is a perspective view of the equipment in FIG. 16 but without an air humidifier, showing the connection structures uncovered;

FIG. 17b is a perspective view of a structural part of the second module of the apparatus of FIG. 17a;

FIG. 17c is a perspective view of the structural part of FIG. 17b, showing a first module inserted into the structural part from below;

FIG. 18 is a perspective view of the air humidifier disconnected from the basic unit;

FIG. 19 is a perspective view of the air humidifier with a cap element removed to show the internal structure of the air humidifier;

FIG. 20 is a further perspective view of the air humidifier, shown separated from the basic unit;

FIG. 21 is a perspective view of selected components of the apparatus of an embodiment of the invention, showing the docking of the air humidifier;

FIG. 22 is an enlarged perspective view of the main cap of the air humidifier;

FIG. 23 is a perspective view of the main cap of the air humidifier showing an elastomer partition element and a heating bar device;

FIG. 24 is a fragmentary view of the elastomer partition element;

FIG. 25 is a perspective view of the airway leading into the main cap and the cup portion of the air humidifier;

FIG. 26 is a perspective rear side view of the apparatus according to an embodiment of the invention;

FIG. 27a is a perspective view of the first module according to a preferred embodiment of the invention;

FIG. 27b is a perspective view of the first module in the receiving portion of the second module;

FIG. 28 is a perspective view of the delivery device according to a preferred embodiment of the invention;

FIG. 29 is a perspective view of the blower of the delivery device of FIG. 28;

FIG. 30 is a perspective view of the blower of FIG. 29;

FIG. 31 is an enlarged fragmentary perspective view of the first module of the invention showing a stub element as an acoustic trap;

FIG. 32 is an enlarged perspective view of a pickup device of the first module of an embodiment of the invention;

FIG. 33 is a perspective view of a stub structure of the second module;

FIG. 34 is a schematic illustration showing the modular construction with the first module inserted into the second module;

FIG. 35 is a perspective view illustrating the partial uncovering of the first module by opening or recessing regions of the second module;

FIG. 36 is a sketch in perspective for illustrating one embodiment of the first module having three cubic portions;

FIGS. 37a and 37b are each sketches for illustrating the construction of an insert element for the inlet portion on the suction side of the first module;

FIG. 38 is a further sketch in perspective of the first module;

FIG. 39 is a sketch of a preferred component for furnishing an airway leading from the CPAP device into a humidifying region;

FIG. 40 is a sketch illustrating an air humidifier cap with a detachably integrated heating bar;

FIG. 41 is a sketch illustrating picking up a differential pressure signal that is indicative of the flow of breathing gas; and

FIG. 42 is a sketch illustrating a bottom plate for simultaneously closing off the combined modules at the bottom and furnishing an elastomer adjusting bottom seal.

FIG. 43a is a simplified side view of a device according to an embodiment of the invention, having a hinged second module that includes a lower structural part and an upper structural part;

FIG. 43b is a side view illustrating an open position of the second module of FIG. 43a;

FIG. 43c is a sketch illustrating a modularly dockable air humidifier;

FIG. 44 is a sketch explaining further advantageous features of the upper structural part in an embodiment of the invention, in this case with detachably insertably embodied electrical modules;

FIG. 45a is a sketch illustrating opening portions, embodied in the upper structural part, for making user-control areas of an electrical module accessible;

FIG. 45b is a sketch illustrating a flip display, which forms part of the control module and which can be viewed above the top of the upper structural part at various erected positions;

FIG. 46a is a sketch illustrating one possible design of a joint structure intended for coupling the two structural parts, which makes both pivotable coupling and the removal of the upper structural part from the lower structural part possible;

FIG. 47 is a sketch explaining the construction of a first module that is insertable into the hinged second module of the invention; and

FIG. 48 is a perspective sketch of a first module, which may be coupled to an upper structural module that furnishes the main user control area of the device.

### DETAILED DESCRIPTION OF ILLUSTRATED EMBODIMENTS

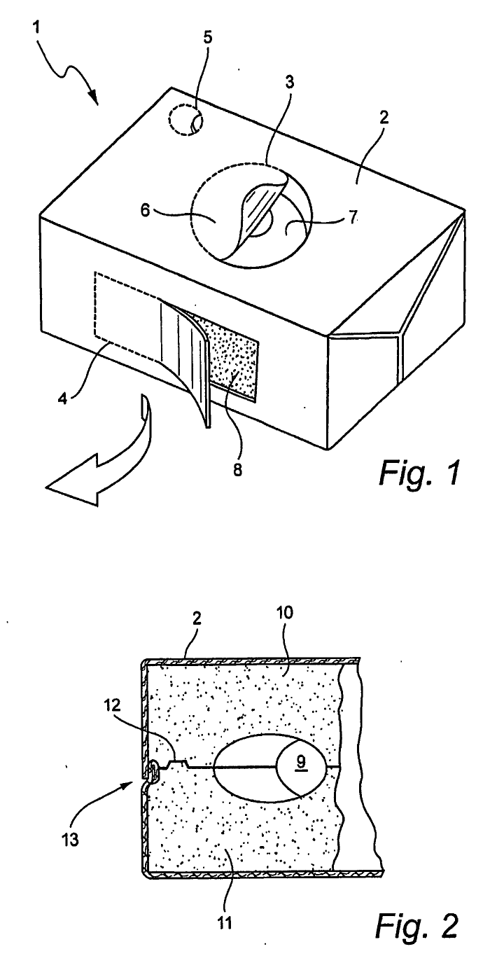

FIG. 1 shows a first module 1 in simplified form, intended for use in a CPAP device. The first module 1 includes a first module housing 2 and sound-absorbing structures located therein. By means of these sound-absorbing structures, an intake-side breathing gas delivery path located upstream of the delivery device and a breathing gas delivery path located downstream of the delivery device are defined.

The first module housing 2 is embodied as a thin-walled structure. This thin-walled structure may in particular be formed of a foil- or film-coated cardboard material, such as the material also known as Tetra-Pak material. The first module housing 2 may be reinforced by the structures received in the first module 1, in particular sound-absorbing structures, so that the first module housing 2 has adequate strength, particularly overpressure strength. The first module housing 2 in this exemplary embodiment is provided with a plurality of perforated plates 3, 4, 5. Via these perforated plates 3, 4, 5, immediately before the first module 1 is used, the opening portions required for operation of a CPAP device can be exposed in the first module housing 2. Via the perforated plate 3, for instance, a wall portion 6 of the first module housing 2 can be torn off, so that a recessed portion 7 intended for receiving a delivery device 16 is exposed.

It is also possible to configure the first module 1 such that it initially has a delivery device 16 (FIG. 4), which can be removed from the first module 1 when the first module 1 is replaced, for instance by first opening an appropriate wall portion of the first module 1 for the purpose.

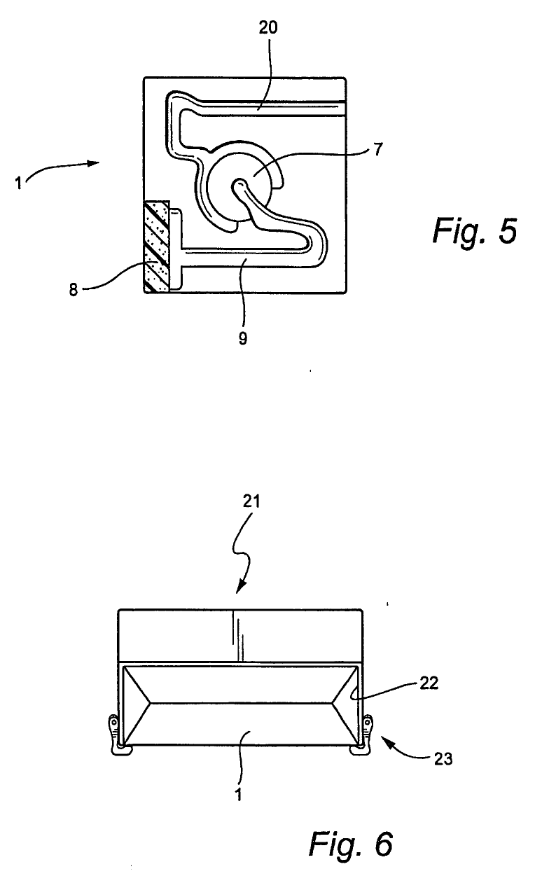

Via the perforated plate 4, in this exemplary embodiment, an opening portion can be exposed in a side region of the first module 1, and breathing gas is delivered via this portion. In the region of this opening portion, as shown in FIG. 5, a foam material portion 8, embodied in particular as an air filter, can be exposed.

Via the perforated plate 5, a compression-side outlet line region of the first module 1 can be exposed. By furnishing the first module 1 as an initially hermetically sealed unit, it becomes possible to prevent any contaminants from getting into the interior of the first module 1. Moreover, when the perforated plates 3, 4, 5 are intact, it is immediately apparent that the first module 1 itself is new and has not yet been used.

Breathing gas conduits adapted to the structural form of the delivery device 16 and to the position of the incoming and outgoing portions of the breathing gas delivery path may be embodied in the first module 1.

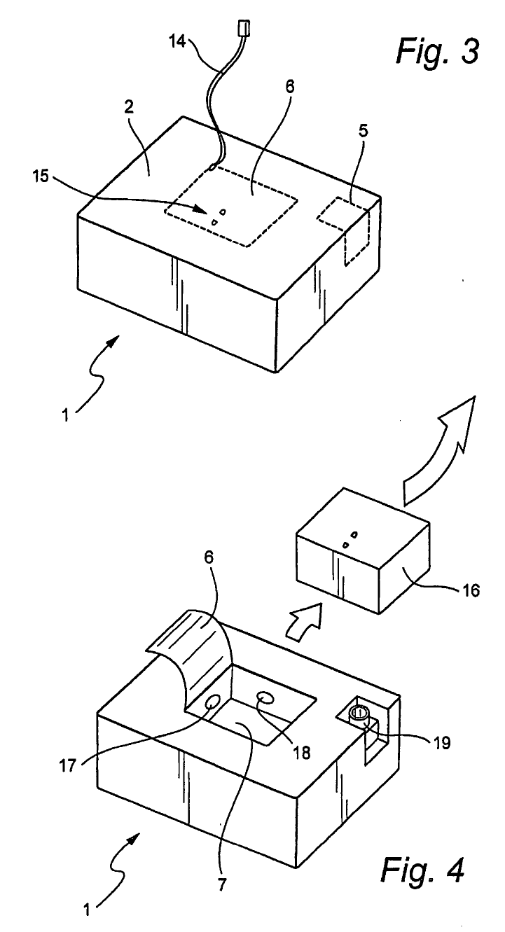

FIG. 2 shows for example how a breathing gas delivery path 9 may be embodied in the interior of the first module 1. In the exemplary embodiment shown here, the breathing gas delivery path 9 is defined by foam insert elements 10, 11, which are positioned relative to one another via positioning structures. The foam insert elements 10, 11 may, as already described above in conjunction with FIG. 1, be inserted into a thin-walled first module housing 2. The course of the breathing gas delivery path 9 is preferably adapted such that while avoiding excessive flow resistance, adequate acoustic absorption effects are attained.

When the first module housing 2 is made of cardboard material, it becomes possible to create folding or crimping zones 13, such that they do not cause any irritating raised areas on the outer surface of the first module 1. It is possible to provide recesses in the foam insert elements 10, 11, so that any folding or crimping protrusions can fit into them.

FIG. 3 shows a variant of the first module 1 of the invention that initially has a delivery device 16 that is already integrated with the first module 1. The power supply to this delivery device 16 is effected in this exemplary embodiment via a flexible line portion 14, which extends as such out of the first module housing 2. It is possible to accommodate the flexible line portion 14 initially such that at first it is not yet exposed to the environment. For instance, the flexible line portion 14 can be located in the region of an indentation or opening that can be exposed via the perforated plate 5. In the case of the first module 1 shown here, it is possible, after the first module 1 has been used, to remove the delivery device 16, initially integrated with this first module 1, by opening a suitable wall portion 6 of the first module 1.

As an alternative to connecting the power supply to the delivery device 16 through the flexible line portion 14 shown here, it is also possible to provide plug structures on the first module 1, in particular socket portions 15 shown in suggested form here, which can be brought into engagement with complementary counterpart plug portions provided on a second module 21. Other kinds of contact concepts may also be utilized to create a disconnectable electrical connection between the first module 1 and a second module 21 (FIG. 6).

In FIG. 4, a first module 1 of the invention is shown in a state in which a wall portion 6, initially covering a receiving portion 7, is disconnected from the remainder of the wall of the first module 1, and the delivery device 16 positioned below this wall portion 6 is removed from the first module 1. As can be seen from this view, the delivery device 16 communicates with corresponding conduit portions 17, 18 that extend in the interior of the first module 1.

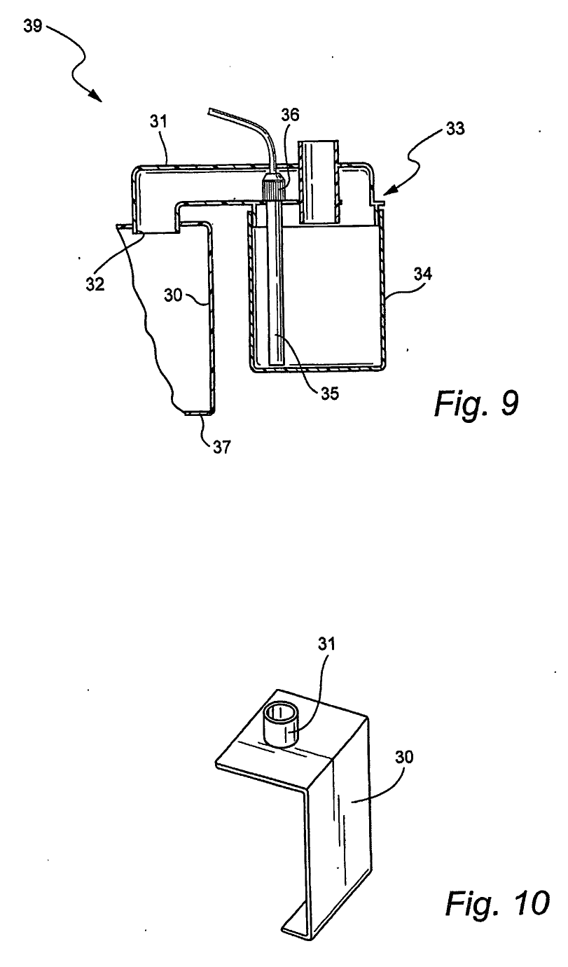

The conduit portion 18, for instance, forms a compression-side portion of the breathing gas delivery path 20 and communicates with a stub portion 19, shown in suggested form here, to which an air humidifier 39 (FIG. 9) or a breathing gas hose may be connected. The stub portion 19 may be located such that, as indicated in FIG. 3, it is initially covered by way of a wall portion defined by the perforated plate 5.

FIG. 5 shows an example of the course that the breathing gas delivery path defined in the first module 1 may have. The delivery path here includes an intake-side breathing gas delivery path portion 9 and a compression-side breathing gas delivery path portion 20. The compression-side breathing gas delivery path portion 20 leads to an outlet line region of the first module 1. The intake-side breathing gas delivery path portion 9 communicates with a filter device 8, formed for instance by a foam material portion. The two breathing gas delivery path portions 9, 20 extend respectively to and from a recessed portion 7, in which a delivery device 16 can be received. The course of the breathing gas delivery paths is shown here merely as an example, variations in the breathing gas delivery path in the first module 1 are encompassed within the scope of the invention. However, the design of the breathing gas delivery paths preferably provides the greatest possible acoustic absorption with the least possible flow resistance. It should be noted that in cooperation with these breathing gas path portions, measurement routes may be implemented, to detect differential pressure signals to provide a signal indicative of the breathing gas flow.

FIG. 6 shows a highly simplified view of the way in which the first module 1 of the invention may be received in a second module 21. The second module 21 in this exemplary embodiment is embodied such that it forms a receiving portion 22, open at the bottom, into which the first module 1 can be inserted. The second module 21 may be embodied such that the functional docking of the first module 1, and the electrical coupling of the delivery device 16, preferably located in the first module 1, is accomplished when the first module 1 is inserted into the second module 21. The first module 1 may optionally be fixed in the receiving portion 22 by an additionally provided fastener 23. The fastener 23 is embodied here as hinged hooks, for example, which simultaneously function as feet for the device to stand on.

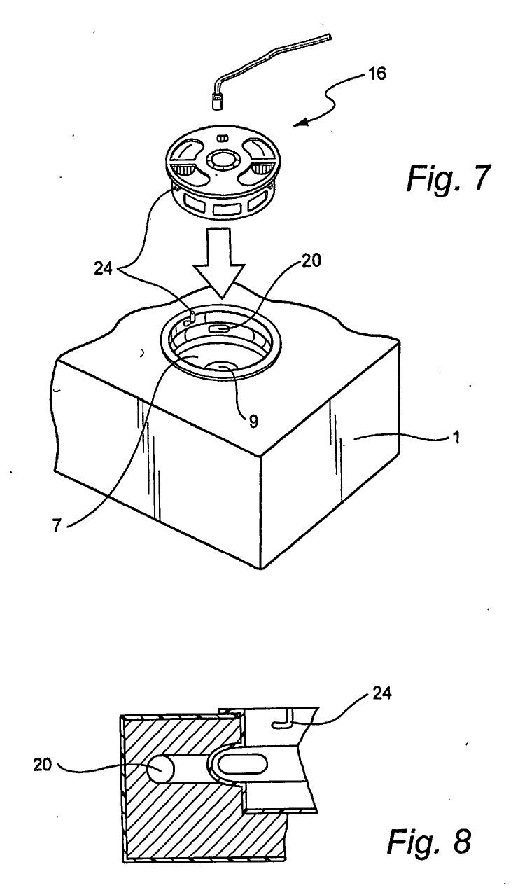

In FIG. 7, a portion of a variant of the first module 1 is shown, which has a cup-shaped receiving portion 7 into which a delivery device 16 may be inserted. The delivery device 16 may be locked in the receiving portion 7 for example using a bayonet mount 24. However, other forms of retaining the delivery device 16 in the receiving portion 7 are encompassed within the scope of the invention. When inserted in the receiving portion 7 the delivery device 16 is in sealed fluid communication with a discharge region of the intake-side breathing gas delivery path 9 and with a beginning region of the compression-side breathing gas delivery path 20.

FIG. 8 shows a sectional view of how a receiving portion 7, intended to receive the delivery device 16, may be furnished by a molded part, preferably made from a recyclable plastic material, in particular styrene.

FIG. 9 in simplified form shows an air humidifier device 39 intended for connection to a first module 1 of the invention. The air humidifier device 39 includes a retaining bracket 30, and a line stub 31 embodied integrally with the retaining bracket. The retaining bracket 30 may, as FIG. 11 shows, be attached to the first module 1 in such a way that a discharge portion 32 of the line stub 31 is seated in sealing fashion in a corresponding outlet discharge portion of the first module 1. The retaining bracket 30 and the discharge portion 32 make it possible to fix the line stub 31 adequately to the first module 1.

The line stub 31 may be embodied in one piece with other functional components of the air humidifier 39, or it may be embodied as a plug-in stub, to which the other airway portions of the air humidifier 39 can be connected. In the exemplary embodiment shown here, the line stub 31 is embodied integrally with a cap portion 33 of the air humidifier 39. A cup element 34 for receiving humidifying water is placed in a sealing fashion against this cap portion 33. The cup element 34 is dimensioned to have a bottom spacing from a surface on which the equipment stands on, making it possible to remove the cup element 34 from the cap portion 33 from below without lifting the CPAP device.

The air humidifier 39 shown here includes a heating bar element 35, which is coupled in sealing fashion to the cap portion 33. The heating bar element 35 is embodied such that it can be disconnected from the cap portion 33 in a simple way, without tools. The plastic parts shown here, that is, the cup element 34, the cap portion 33, and preferably the line stub 31 that is integral with the cap portion 33, may be embodied as disposable components, which can be replaced or disposed of after a certain length of time in use, or after use by a single patient. The heating bar element 35 is preferably embodied such that it can be used multiple times. The heating bar element 35 is inserted, for instance via an elastomer stopper 36, through the cap portion 33 into the cup element 34 in a sealing manner.

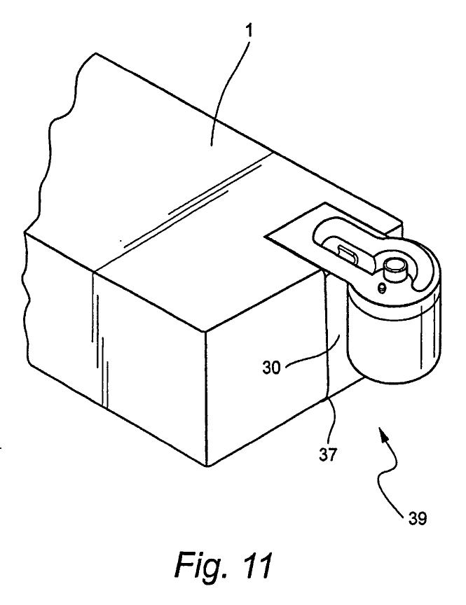

In FIG. 10, a variant of the retaining bracket 30 is shown, in which the line stub 31 is embodied as a short tubular peg that protrudes upward in the vertical direction. A corresponding connection portion of a breathing gas hose can be slipped, optionally directly, onto this line stub 31.

FIG. 11 shows how the air humidifier device 39 of FIG. 9 may be attached to a first module 1 of the invention. The fixation of this air humidifier device 39 is done using the retaining bracket 30. The retaining bracket 30 has an angled portion 37 that engages the bottom surface of the first module 1 diametrically opposite the line stub 31 (see FIG. 9).

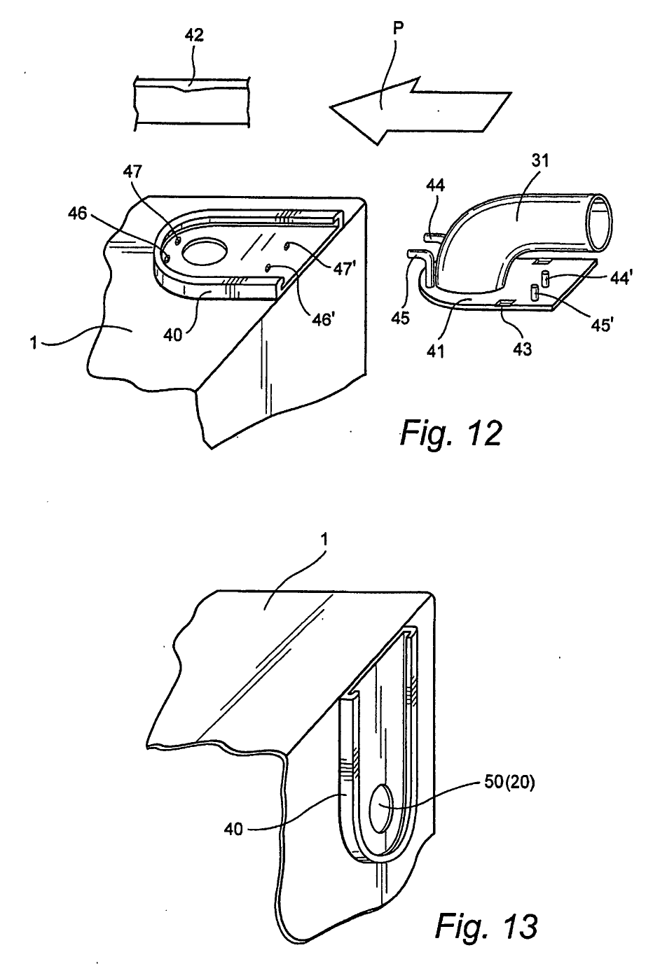

FIG. 12 shows a further variant for accomplishing the connection of a line stub 31 to the first module 1. In this embodiment, a receiving structure 40 is provided on the first module 1, and a connection plate 41 of the line stub 31 can be thrust into this receiving structure, as indicated by the arrow symbol P. Fixation structures 42, 43, may be provided on both the receiving structure 40 and the connection plate 41 to sufficiently lock the connection plate 41 in the receiving structure 40.

Using the receiving structure 40 and the connection plate 41a pressure measurement conduit system may be defined to obtain a differential pressure signal indicative of the breathing gas flow. This pressure measurement conduit system may have pressure measurement line portions 44, 45 and 44′, 45′, respectively, which in the joined position of the connection plate 41 communicate with pressure measurement conduits 46, 47 and 46′, 47′, respectively. The pressure measurement conduits 46, 47 are extended in the interior of the first module 1 in such a way to allow measurement of a differential pressure signal, which is in a largely linear relationship with the flow of breathing gas, within the breathing gas delivery path.

The pressure measurement conduit stubs 44, 45 may be located such that when the line stub 31 is attached or the connection plate 41 is inserted, they enter directly into engagement with corresponding pressure pickup conduit portions of the first module 1.

In FIG. 13, a variant of the receiving structure 40 is shown, wherein a line stub 31 (not shown) is connectable to a vertical wall of the first module 1. A connection plate 41 or a line flange portion can be thrust into this receiving structure 40, similarly to the embodiment of FIG. 12, so that a corresponding line stub communicates in a sealing fashion with the portion of the breathing gas delivery path 20 that is exposed by way of the orifice 50 that can be seen here.

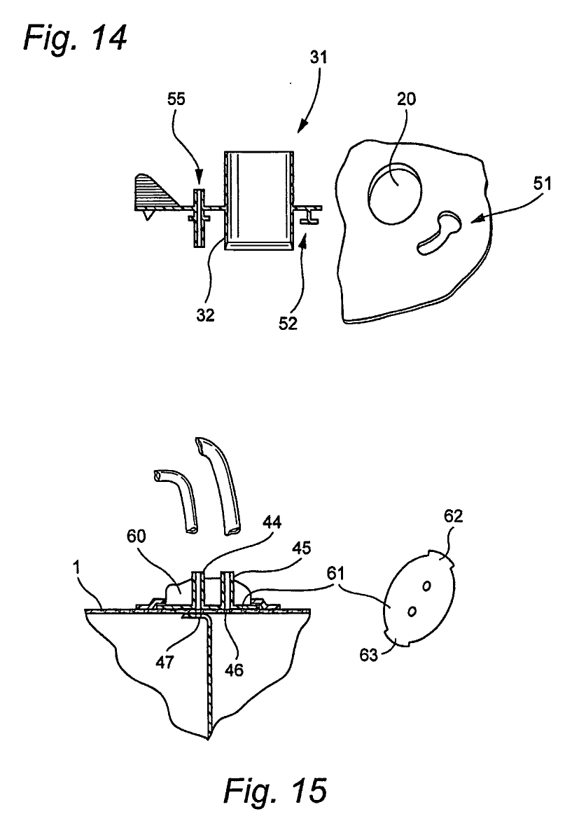

In FIG. 14, a further exemplary embodiment for a line stub 31 is shown. This line stub 31 may be inserted by a lower orifice portion 32 into a corresponding orifice or air outlet opening of a breathing gas delivery path portion 20 and may be fixed via the bayonet mount structure 51, 52 as shown. Also in this exemplary embodiment, it is possible in the region of the line stub 31 to furnish a pressure measurement conduit 55, by way of which a differential pressure pickup may be made.

In FIG. 15, a pressure measurement adapter is shown, which forms two pressure measurement stubs 44, 45, which can be coupled detachably to the first module 1. The pressure measurement stub 44 communicates with a pressure measurement conduit 47, which discharges into a first portion of the breathing gas delivery path 20. The second pressure measurement conduit stub 45 communicates with a conduit 46, which discharges into the breathing gas delivery path 20 in a second position, which is spaced apart from the first pressure pickup point (47). The pressure measurement adapter 60 may be embodied such that it includes a base plate 62, which is provided with bayonet locking edges 62, 63 and is lockable to the first module 1 by way of them.

In the abovementioned exemplary embodiments, all the hygiene-relevant components of the breathing gas are integrated into a single first module. However, the invention is not limited to this principle. For instance, it is also possible to provide separate (replaceable) delivery path elements for both the intake region of the breathing gas delivery path and the compression region of the breathing gas delivery path (see below for further detail). The delivery device 16 can be embodied in parts such that its electrical components can be disconnected from the components that come into contact with the breathing gas.

The detection of a signal indicative of the breathing gas flow can be made in the form of a differential pressure detection. As an alternative, other kinds of physical interaction effects may be used for generating a corresponding signal. For instance, it is possible to provide an element in the region of the breathing gas delivery path 20 that deforms or bends as a function of the speed of the air flowing past, and for the deformation to be detected optically. For instance, it is possible to provide a flexible waveguide in the region of the breathing gas delivery path 20 that emits a beam of light whose orientation varies as a function of the instantaneous breathing gas flow.

In a first embodiment the first module 1 of the invention may be used in the flowing manner to make a completely sterile CPAP device. Initially a first module 1 of the invention is picked up and prepared suitably for assembly, by detaching one wall portion 4 of the first module 1 that covers an inlet region and one wall portion 5 that covers a breathing gas outlet line region from the first module 1 at perforated places provided for the purpose. The thus-prepared first module is inserted into a receiving portion of a second module 21. In the second module 21, electrical contact elements enter into engagement with contact portions that are embodied in the region of the first module 1. As a result, a current circuit is formed that extends via a blower motor. An air humidifier is now attached to the first module 1 that has now been inserted into the second module 21. Attaching the air humidifier 39 is done by pushing a connection plate 41 of the air humidifier 39 against a receiving structure 40, provided for instance on the first module 1. Docking of the air humidifier 39, or of the breathing gas line portions that lead to the air humidifier 39, makes it possible to couple a differential pressure sensor, provided in the region of the connection plate 41, to pressure pickup points that are provided in the region of the receiving structure 40 preferably provided on the first module 1.

A breathing gas hose may be connected to the air humidifier 39, and a breathing mask can be connected to this hose in a known manner. To this extent, a ready-to-use system for delivering a breathing gas is now attained.

If after this system has been used a need for cleaning arises, the breathing gas hose and the breathing mask may be disconnected from the air humidifier 39 and cleaned, each as separate parts, with a preferably heated cleaning solution, and optionally heat-treated afterward.

The air humidifier 39, and the first module 1 of the invention, may be disconnected from the second module 21 and either cleaned or disposed of. After the first module 1 is removed from the second module 21 the delivery device 16 received in the first module 1 may be removed from the first module 1. The delivery device 16 and optionally the air humidifier 39 may be taken to a system processor and optionally delivered for a further use. The other components of the first module 1 may be disposed of.

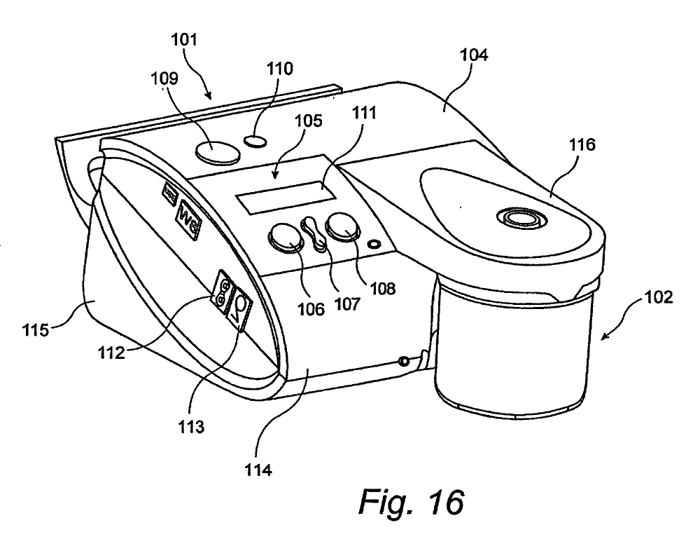

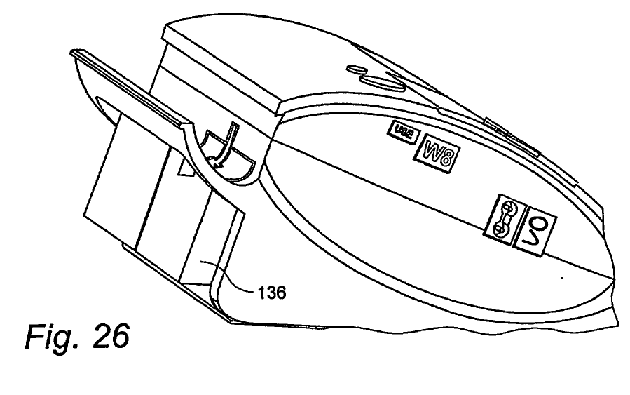

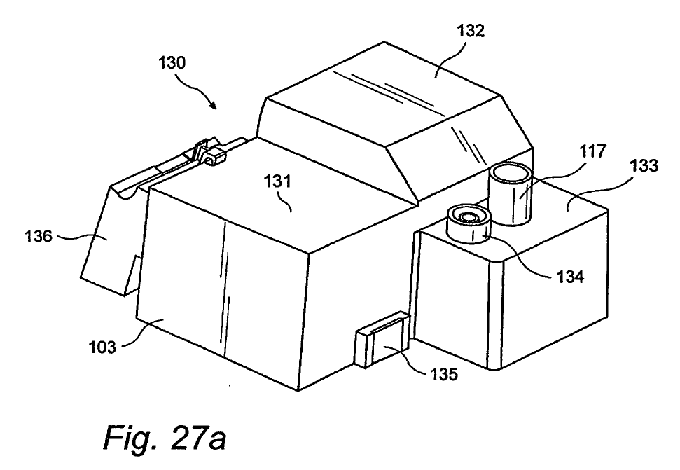

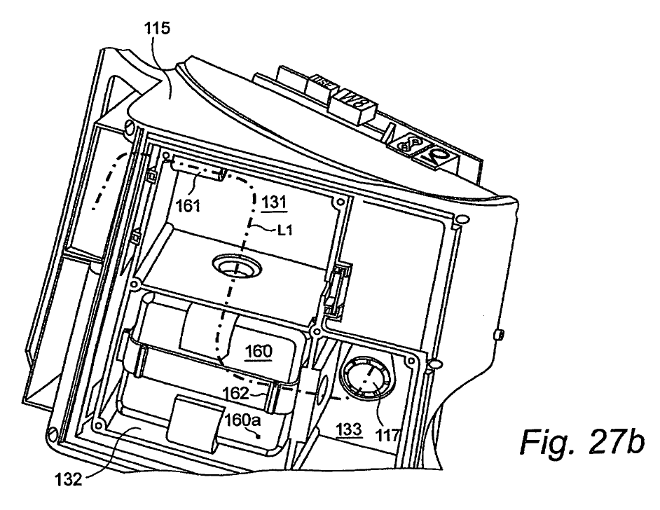

FIG. 16 shows a piece of equipment according to a further embodiment of the invention for furnishing breathing gas, particularly ambient air, at a pressure level that is at least in some phases above the ambient pressure. The equipment shown is of modular construction and includes a basic unit 101 with an air humidifier 102 detachably coupled to it. The basic unit 101, as will be further explained hereinafter, is of modular construction and includes a first module 103 (see FIG. 27a), not yet visible in this view, and a second module 104, which also forms the outer housing 114. The first module 103, which in this view is completely concealed by the second module 104, includes the delivery path components that serve to form a breathing gas delivery path. The second module 104 includes the further apparatus components, such as the electronic circuit devices in particular, not further shown here, that can be separated from these delivery path components. The second module 104 is provided with an operator control panel 105 and with a plurality of switching panel elements 106, 107, 108, 109 and 110, and a display device 111. The operator control panel 105 may be embodied such that it can advantageously be adapted to different equipment configurations and different country requirements. It is possible to design the operator control panel 105 such that in the usual usage state, it has a surface that can advantageously be operated intuitively; switching devices for achieving more-complex equipment configurations are acceptable particularly once a cap device of the operator control panel has been removed. It is possible to construct the equipment such that it is given certain configurations as a function of different types of cap device. For instance, different configurations for different countries can be achieved by means of different types of cap device.

An electrical outlet device 112 for connecting a power cord and a mains switch 113 are provided on the second module 104. The mains switch 113 and the outlet 112 can be combined into a subsidiary group (mains structural unit). The electrical outlet device 112 is embodied and dispensed such that any contaminants penetrating it, or water penetrating it, can advantageously fall out or flow out of this electrical outlet device 112. The electrical outlet device 112 and the mains switch 113 are integrated into an upper structural part 114 of the second module 104. As a result, the electrical connection between the electrical components coupled to the upper structural part 114 can be maintained even after the upper structural part 114 has been detached from a lower housing part 115 of the second module 104.

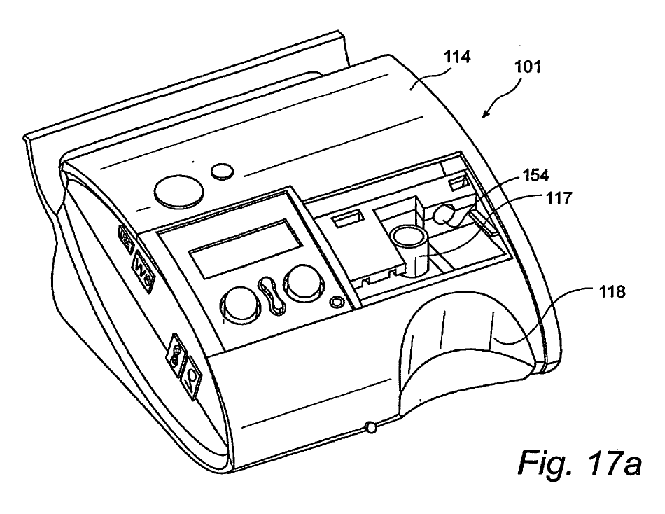

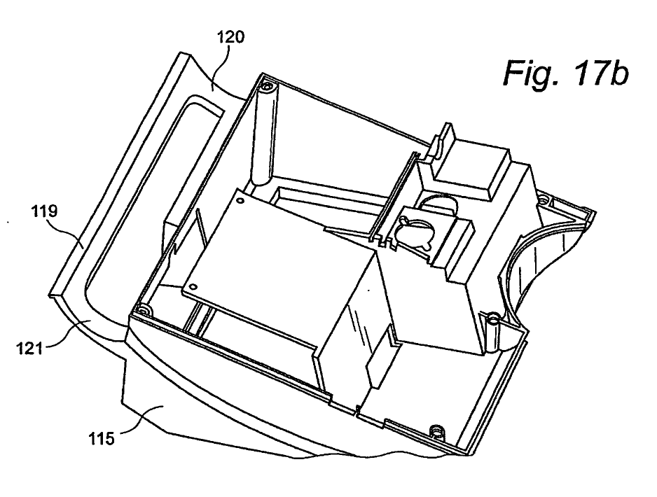

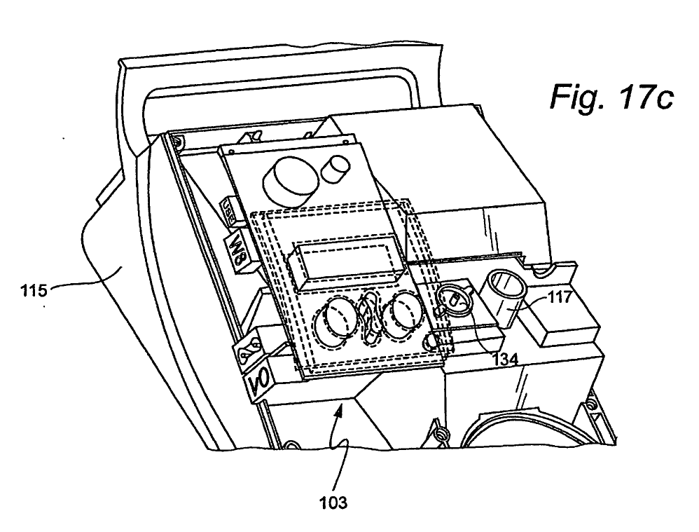

The lower housing part 115 is a structural part that forms a receiving portion for receiving the first module 103 shown in FIG. 27a. The first module 103 and the second module 104, in the assembled state, are coupled with one another such that certain portions of the first module 103 at least partially penetrate the second module 104 in the region relevant for coupling the air humidifier 102, or are in the open through the second module 104 (see FIG. 17a).

The air humidifier 102 is coupled to the basic unit 101 using a cap element 116, which is pivotably and detachably connected to the upper structural part 114 of the basic unit 101 via a hinge device (not shown). Flipping the cap element 116 open makes it possible to remove the actual components of the air humidifier 102 from the basic unit 101. In this embodiment, however, the cap element 116 not only serves as a cladding element but also serves to lock the actual air humidifier 102 to the basic unit 101.

FIG. 17a shows the basic unit 101 of the equipment of FIG. 16 with the air humidifier 102 removed. As can be seen from this view, a connection stub 117, which is part of the first module 103 (see FIG. 27a), forms an air humidifier connection region that is otherwise concealed by the cap element 116 shown in FIG. 16. It is possible for a flexible breathing gas hose, provided for carrying the breathing gas supplied by the basic unit 101 onward, to be connected directly to this connection stub 117 and in this way to operate the equipment without an air humidifier 102. Preferably when the basic unit 101 is operated without an air humidifier 102 attached a covering element (not shown) is placed over the exposed structures. The covering element may also cover the receiving recess 118, advantageously formed in the basic unit 101 to receive the cup portion of the air humidifier. The upper structural part 114 is provided with suitable fasteners to allow attachment of either an air humidifier 102 or the cover element, allowing configuration of the equipment with or without an air humidifier as appropriate. The connection stub 117 is oriented such that its axis is oriented essentially vertically and in particular extends perpendicular to the surface the equipment stands on.

FIG. 17b shows the lower housing part 115 of the second module 104. This lower housing part 115 is provided with a handle 119, which serves to make it easier to manipulate the basic unit 101. The handle 119 forms a grip crosspiece, which at lateral fastening portions 120, 121 merges with the main structure of the lower housing part 115. The fastening portions 120, 121 are curved in such a way that by means of the handle 119, a support structure is furnished on which a portion of a breathing gas hose can be placed (not shown).

The lower housing part 115 forms three main receiving portions, accessible from below, for receiving the first module shown in FIG. 17c. The lower housing part 115 also forms positioning and fixation structures (not indicated) for the correct positioning of the first module 103 relative to the second module 104, of which the lower housing part 115 also forms a component.

FIG. 17c indicates the positions of the connection stub 117 and the measurement stub structure 134 when the first module 103 is inserted into the receiving chamber of the lower housing part 115 shown in FIG. 17b. The measurement stub structure 134 is located here at a point through which the pressure measuring connections, furnished by the measurement stub structure 134, are located in the immediate vicinity of a circuit board element (not shown) and can be coupled either directly to a pressure converter provided on this circuit board element or optionally to the pressure converter with the interposition of two short flexible hose segments.

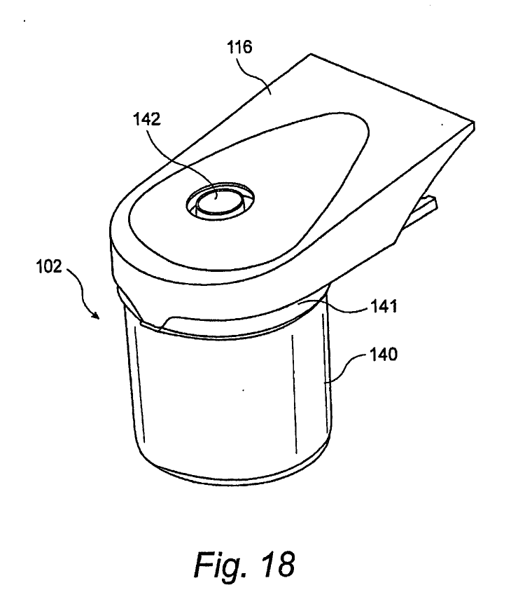

FIG. 18 shows the air humidifier 102 of the invention, together with a cap element 116 that is effective as both cladding and as a fastening device. The air humidifier 102 includes a cup portion 140 and an air humidifier main cap 141, coupled detachably and in sealing fashion to this cup portion 140. The cup portion 140 receives the water that may be required to humidify the breathing gas. The cup portion 140 is preferably made at least in some portions of a transparent or translucent material, for easier monitoring of the fill level. The air humidifier 102 is constructed such that the filling of the cup portion 140 can be done either after the air humidifier is removed from the basic unit 101 and after removal of the main cap 141, or optionally merely after removal of a breathing gas hose (not shown) from a breathing gas hose connection stub 142. In terms of its volume, the cup portion 140 is dimensioned to hold a sufficient quantity of water for humidifying the breathing gas over a typical administration time of approximately six hours. The main cap 141 of the air humidifier 102 is coupled to the cup portion 140 in such a way that the cup portion 140 cannot be disconnected from the main cap 141 of the air humidifier 102 during the delivery of typical breathing gas pressures. Preferably to ensure particularly secure coupling of the cup portion 140 to the main cap 141 of the air humidifier 102 bayonet-mount-like coupling structures may be employed on the cup portion 140 and on the main cap 141 of the air humidifier 102. It is also possible to embody the air humidifier 102 such that a sufficiently firm coupling between the cup portion 140 and the main cap 141 of the air humidifier 102 is attained in cooperation with structures that are embodied on the basic unit 101 and that become operative after the air humidifier 102 has been docked to the basic unit 101.

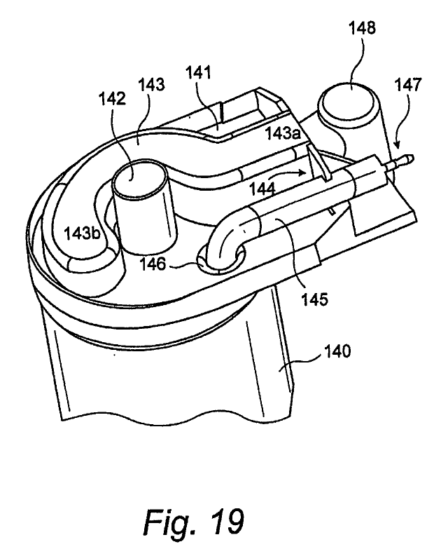

In FIG. 19, the main cap 141 of the air humidifier 102 is shown in further detail. The main cap 141 of the air humidifier 102 includes a breathing gas line portion 143, which extends from an inlet portion 143a to an outlet portion 143b that leads into the cup portion 140. The breathing gas line portion 143 is curved in crescent-like fashion and thus extends around the central breathing gas outlet stub 142 located above the cup portion 140. This arrangement provides good protection against a return flow of humidifying water into the basic unit 101.

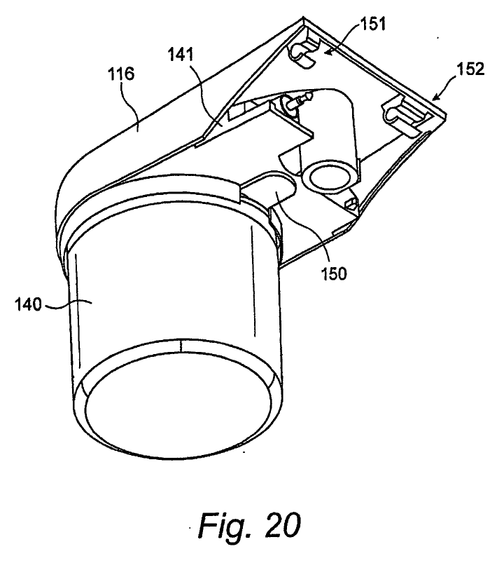

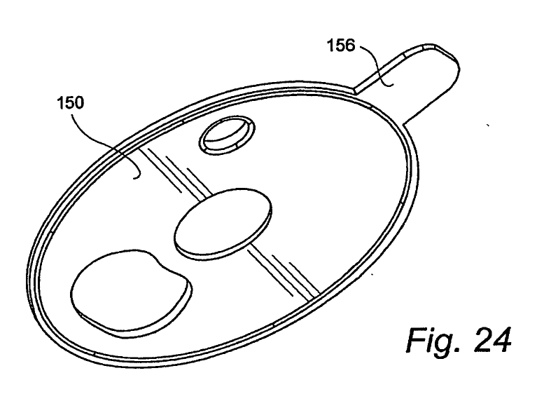

The main cap 141 of the air humidifier 102 forms a fastening structure 144 for fixation of a heating bar 145. In this embodiment, the heating bar 145 is bent over and is coupled detachably to the main cap 141 of the air humidifier 102 via the fastening structure 144. The heating bar 145 is extended in sealing fashion through the main cap 141 of the air humidifier 102. The sealing is accomplished by a duct element 146, which is made of an elastomer material and which, as will be described in further detail hereinafter, forms an integral component of a partition element 150 (see FIGS. 20 and 24), by which the breathing gas line portion 143 is largely covered toward the inner region of the cup portion 140.

The heating bar 145 is provided with an electrical connection element 147, which upon docking of the air humidifier 102 to the basic unit 101 shown in FIG. 17a are coupled directly to complementary connection devices located within a recess 154 provided on the basic unit 101.

The angle stub 148 is fundamentally not a component of the air humidifier 102 but connects the air flow from the basic unit 101 to the air humidifier 102. The angle stub 148 is placed on the connection stub 117 of the first module 103 (see FIG. 17a). The placement of the angle stub 148 on the connection stub 117 occurs prior to the docking of the air humidifier 102 to the basic unit 101. The angle stub 148 is designed such that maintains the prevention of any return flow of water from the air humidifier 102 into the first module 103.

FIG. 20 shows the under side of the air humidifier 102 of FIG. 19 indicating the under side of the cup portion 140 and the main cap 141. The cup portion 140 is coupled to the main cap 141 of the air humidifier 102 via a threaded or bayonet-mount structure. The sealing is supported by the partition element 150, shown separately in FIG. 24. As shown, a hinge structure 151, 152 may detachably pivotably connect the cap element 116 to the basic unit 101 shown in FIG. 17a. The positionally correct fixation of the air humidifier 102 to the basic unit 101 (FIG. 17a) may be reinforced by further locks and fasteners, embodied particularly on the main cap 141 of the air humidifier and optionally also on the cup portion 140.

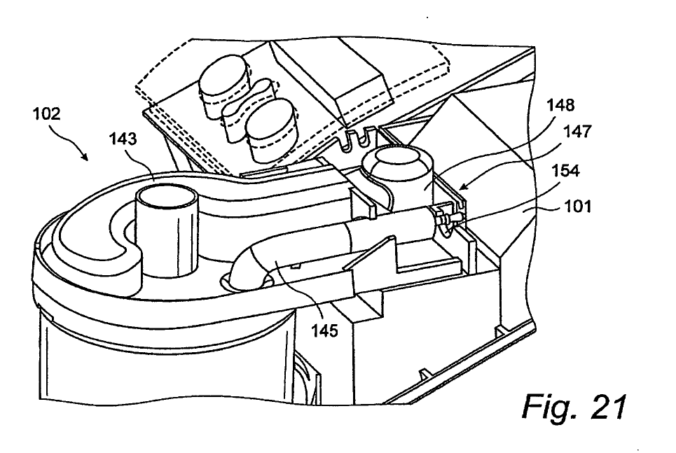

FIG. 21 indicates the position that the corresponding components of the air humidifier 102 and of the basic unit 101 assume relative to one another in the assembled state. The breathing gas line portion 143, of the main cap 141 of the air humidifier 102, is connected to the connection stub 117 (see FIG. 17a) of the first module 103 by using the angle stub 148. The electrical connection elements 147 embodied on the heating bar 145 may be coupled to an electrical connection device (not shown), located in a recess 154 of the lower housing 115.

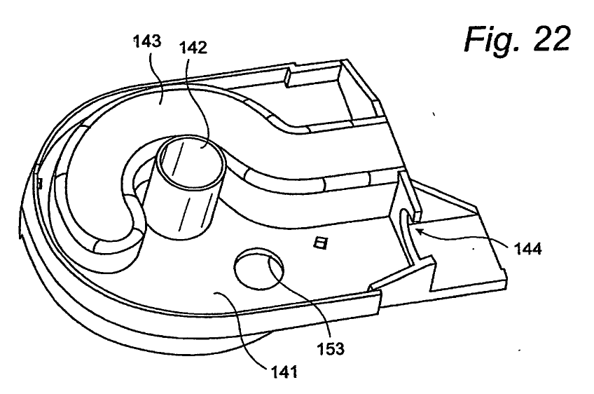

FIG. 22 shows a detailed perspective view of the main cap 141 of the air humidifier 102. The breathing gas connection stub 142 is adapted to connect to a breathing gas hose, or a plug device associated with such a breathing gas hose. Variations in the construction of the breathing gas connection stub 142 are also encompassed within the scope of the invention.

The opening 153 embodied in the main cap 141 of the air humidifier 102 serves as a passage for the heating bar 145, as shown in FIG. 19. The heating bar 145 (FIG. 19) can be detachably introduced into the fastening structure 144 shown here as a fixation claw, formed on the main cap 141 of the air humidifier 102, and fixed sufficiently well in this position.

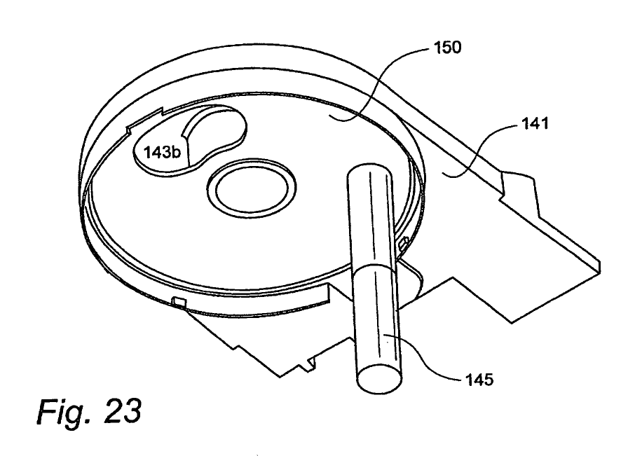

The breathing gas line portion 143 of the main cap 141 of the air humidifier 102 predominantly forms a channel, which is open toward the underside of the main cap 141 of the air humidifier 102 and is thus easy to clean. As can be seen from FIG. 23, this channel can be covered by the partition element 150 in such a way that an entry of the breathing gas into the cup portion takes place only via an opening embodied in the partition element 150 in the region of the outlet portion 143b of the breathing gas line portion 143. The partition element 150 is made from an elastomer material and at the same time forms a sealing device for the sealed passage of the heating bar 145 through the main cap 141 of the air humidifier 102 and also forms a ring seal for sealing off the peripheral circumferential region of the cup portion 140 (FIG. 19) that can be connected to the main cap 141 of the air humidifier 102.

FIG. 24 shows a preferred construction of the partition element 150, as it can be used with the main cap 141 of the air humidifier 102 described above. The partition element 150 is provided with a tab 156, which makes it simple to remove the partition element 150 from this main cap 141 of the air humidifier 102 in order to clean the main cap 141 of the air humidifier 102.

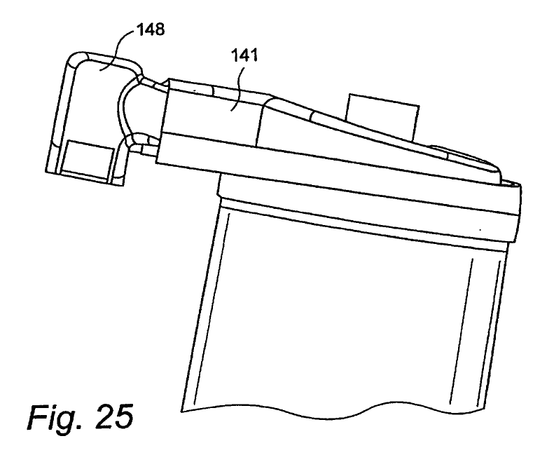

FIG. 25 shows a side view of the air humidifier 102 indicating how the main cap 141 cooperates with the angle stub 148. The angle stub 148 may be made from an elastomer material or from a relatively dimensionally stable material and may be provided with appropriate sealing rings, seated for instance in O-ring grooves.

FIG. 26 shows a perspective view of the rear wall region of the basic unit 101. In this view, the covering structure 136 that is accessible from outside can be seen particularly well; it covers an entry region of the first module 103 (see FIG. 17a). This covering structure 136 is shown again in FIG. 27a. As indicated there, a foam body or other kind of body made from a filter material can be seated beneath this covering structure.

In FIG. 27a, the first module 103 is shown, which serves to receive the delivery path components that define the breathing gas delivery path. The first module 103 includes a main body 130 with an inlet portion 131 on the suction side, a delivery device receiving portion 132 serving to receive the breathing gas delivery device, and an outlet portion 133. The outlet portion 133 comprises the connection stub 117, which has been discussed above in conjunction with FIG. 17a. In the constructed state the connection stub 117 penetrates an opening provided in the second module 104. A measurement stub structure 134 is also embodied on the outlet portion 133, and by way of the measurement stub structure 134, a differential pressure signal, picked up in the region of the breathing gas outlet housing portion 133, can be delivered to a differential pressure converter provided in the region of the second module 104. The first module 103 shown here may optionally be provided with electrical components as well, in particular a recording device 135, by way of which data indicative of the use, such as the length of time the first module 103 has been used, can be recorded. Instead of the recording device 135, or in combination with it, linking elements may also be embodied, preferably at the position shown here, for creating an electrical link up with the electrical components constructed in the region of the first module 103, in particular the electric motor of a blower device, to be described in further detail hereinafter.

A covering structure 136 may also be coupled to the first module 103, and through it the breathing gas inlet region, not visible here in detail but furnished through an opening in the component 130, can be covered. A filter device, in particular a filter made from a foamed plastic material, can be replaceably disposed in the region of the covering structure 136.

The first module 103 includes the three main portions 131, 132 and 133 as mentioned above. These main portions each preferably have a cubic construction. The connection stub 117 is seated on the outlet portion 133 and is oriented such that in the position for use it extends substantially vertically. The measurement stub structure 134 is also embodied such that it rises, essentially parallel to the center axis of the connection stub 117, from the outlet portion 133.

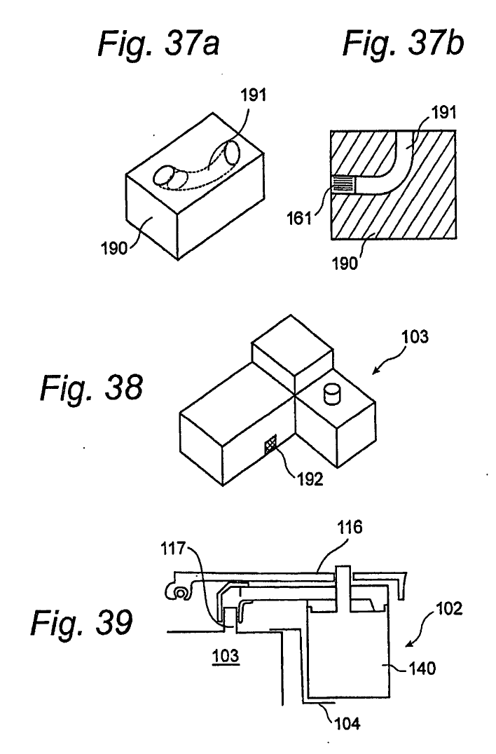

In FIG. 27b, a partially disassembled first module 103 is shown in the state in which it is inserted into the lower housing part 115. The first module 103 comprises the inlet portion 131, the delivery-device-receiving portion 132 intended for receiving the delivery device 160, and the outlet portion 133 that discharges into the connection stub 117. In the inlet portion 131 and the breathing gas outlet housing portion 133, insert elements 190 (see FIGS. 37a and 37b) made from a foamed material are provided, which define a breathing gas conduit that is preferably curved only slightly, and most preferably only include a single curve, extend between the various openings in the applicable portion. In the region of the inlet portion 131, a stub element 161 is provided, which from an inlet opening in the inlet portion 131 protrudes approximately 30 mm into the interior of the inlet portion 131. This stub element 161 creates an acoustic trap, which largely prevents any running noises generated by the delivery device 160 from being emitted into the environment.

The delivery device 160 is received in a housing 160a, which is preferably made from a material with a relatively high specific weight, in particular metal. The housing 160a in this exemplary embodiment is braced via an elastomer structure 162 in the delivery device receiving portion 132. The elastomer structure 162, as will become clear below in conjunction with FIG. 28, also simultaneously seals and connects a blower when the blower is received in the housing 161.

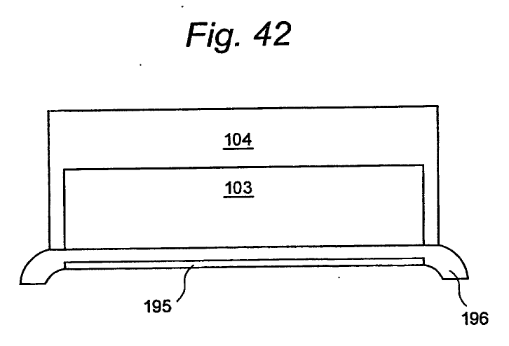

The first module 103 is constructed to minimize the number of deflection points created in the breathing gas path. As a result, the flow resistance of the breathing gas path is reduced considerably, compared to conventional constructions. The course of the breathing gas path is approximated here by the dot-dashed line LI. As already noted, in the inlet portion 131 and outlet portion 133, there are preferably foam insert elements 190 (see FIGS. 37a and 37b), also serving to absorb sound, into which the conduits surrounding the dot-dashed line LI are molded. The bottom region of the first module 103 is preferably covered by a bottom seal 196 (see FIG. 42), which is made from an elastomer material. This bottom seal 196 can be shaped such that it also covers the lower bottom region of the lower housing part 115. A bottom plate 195 (see FIG. 42) made from a preferably more-rigid plastic material can also be screwed onto this bottom seal. The use of this bottom seal 196 advantageously elastically or vibrationally dampens the equipment when positioned on a tabletop.

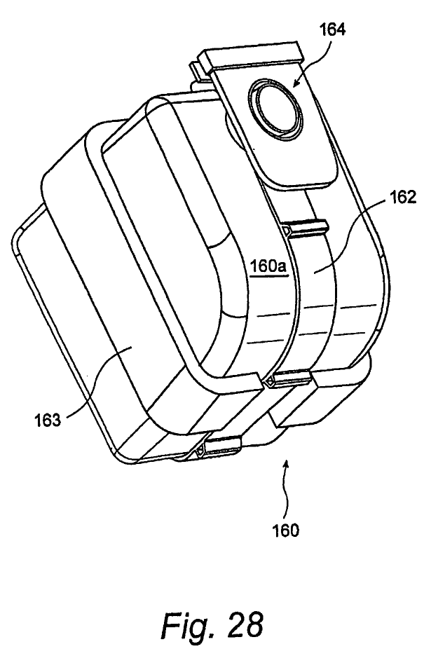

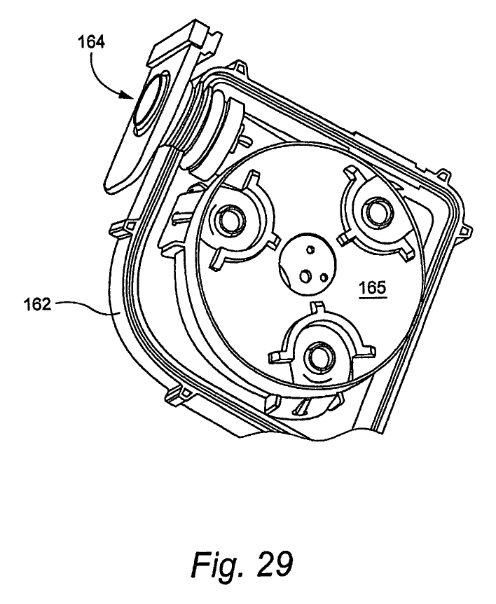

In FIG. 28 the delivery device 160 is shown, inserted into the delivery device receiving portion 132 of the first module 103. The delivery device 160 includes a housing 160a embodied in two parts and an elastomer structure 162 that both couples the two halves of the housing 161 together and supports the blower 165 (see FIG. 29), which is located in the inner region of the housing 160a. The housing 160a is also surrounded by a sheath 163 made from a foam material. The elastomer structure 162 forms a connection conduit portion 164, by way of which the air flow course located downstream of the blower 165, to be described hereinafter, is guided into the outlet portion 133 of the first module 103.

In FIG. 29, the construction of the elastomer structure 162 is shown further. This elastomer structure 162 provides both support to a blower 165 within the housing 160a and an extension of the blower conduit on the pressure side out of the housing 160a.

The blower 165, as shown in FIG. 30, may be additionally braced in the inner region of the housing 160a by further foam structures 166. By means of these foam structures, the air flow course of the air in the interior of the housing 160a can also be varied. The construction of the delivery device 160 shown as an intrinsically modularly closed unit makes it possible for blower devices 165 of different construction to be disposed in the interior of the housing 160a, making an especially advantageous compatibility with the various blower device constructions available.

FIG. 31 shows the inlet portion 131 located on the suction side relative to the delivery device 160, along with the stub element 161 protruding into this inlet portion 131. A main voltage connection structure 170 may be provided in the inlet portion 131 to allow supply of the voltage to the delivery device 160.

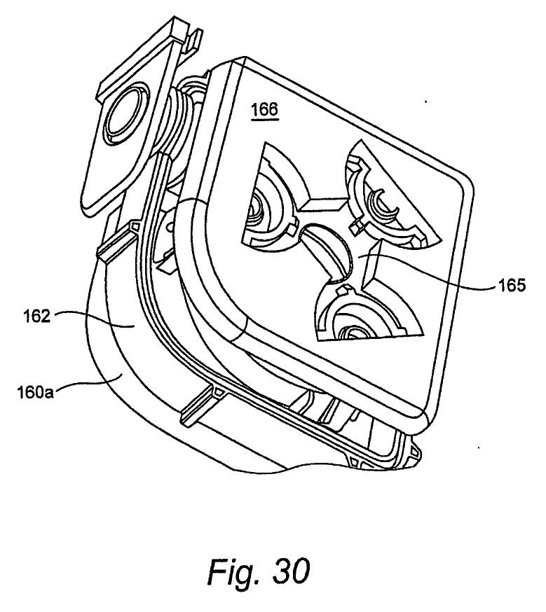

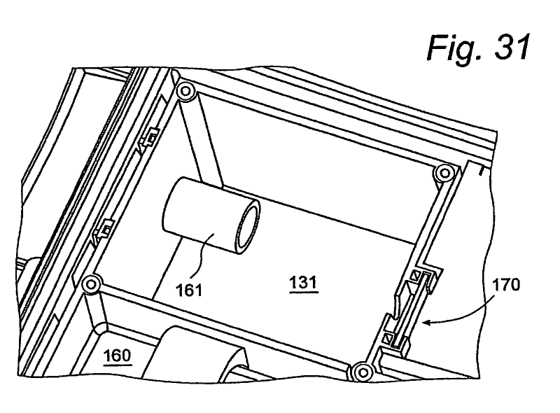

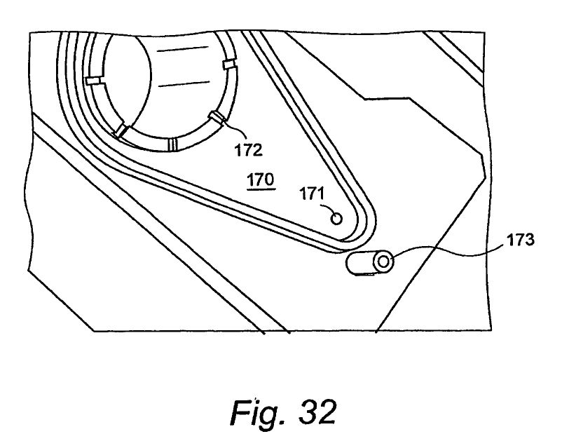

FIG. 32 shows a preferred embodiment of a pressure pickup device located in the region of the outlet portion 133, particularly in cooperation with the housing of the first module 103. The indentation 170 visible here forms a connecting chamber by way of which a first differential pressure measuring conduit 171 communicates with the measuring caverns 172. The measuring caverns 172 are distributed uniformly about the connection stub 117. They connect the inner region of the connection stub 117 to the interior defined by the indentation 170. This interior is covered by a flat covering, not shown here, and is separated by it from the rest of the chamber. The pressure pickup from this chamber is made via a second differential pressure measuring conduit 173.

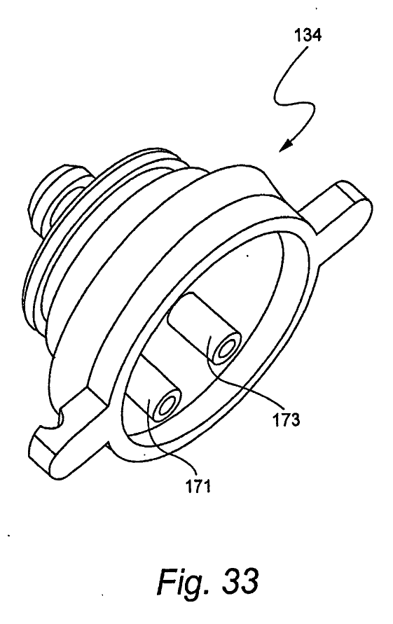

The pressures picked up via the two differential pressure measuring conduits 171, 173 can advantageously be applied, via the measurement stub structure 134 shown in FIG. 33, to a circuit device located in the region of the second module 104.

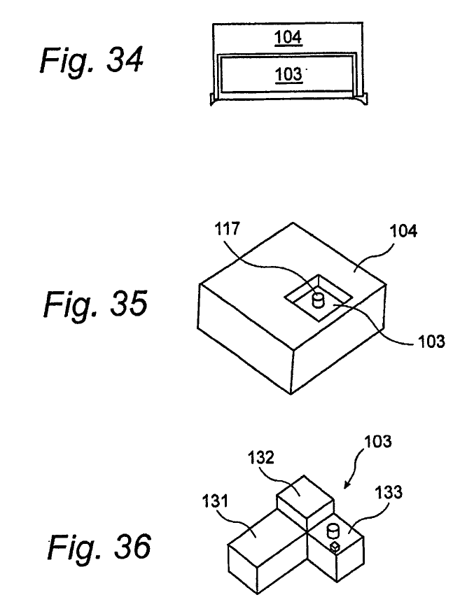

FIG. 34 schematically shows the modular construction of the basic unit 101. The basic unit 101 includes one group, referred to as the first module 103, that is relevant to soiling, and one operator control or electronic group, referred to as the second module 104. The second module 104 includes a housing structure, in which the first module 103 is received in at least some portions.

In FIG. 35, the first and second modules 103,104 are combined, in such a way that in some portions they penetrate one another, or in particular a connection stub 117 of the first module 103 is in the open, via a region defined by the second module 104.

FIG. 36 schematically shows the first module 103, broken down into an inlet portion 131, a delivery device receiving portion 132, and a outlet portion 133. The first module 103 includes a body in which the three portions 131,132,133 and the delivery device 160 are located. The delivery device 160 is inserted, again as a replaceable subsidiary component group, into the body.

FIGS. 37a and 37b show an insert element 190 in simplified form. The insert element 190 is made from a foamed material and defines a breathing gas path portion 191. The breathing gas path portion 191 has a course curved only once, for the sake of attaining a slight flow resistance. In this respect, a departure from the previously conventional labyrinth attachments is made. On the inlet side, a stub element 161 protrudes into the breathing gas path portion 191, and this stub element 161 forms an acoustic trap in cooperation with the chamber wall that receives the insert element 190.

In FIG. 38, a recording device 192, such as a memory card, is provided on the first module 103 and contains for instance soiling-relevant information, length of use, frequency of use, or information about technical properties of the delivery device 160. This information can be detected and taken into account by the second module 104.

FIG. 39 illustrates the locking of the air humidifier 102 to the basic unit 101. The connection stub 117 is furnished by the first module 103. The structure for the equipment to stand on and receiving the container portion 140 is furnished by the second module 104. Locking of the air humidifier 102 is effected by means of a butterfly-valve and cap element 116. The butterfly-valve and cap element 116 is detachably mounted on the basic unit 101.

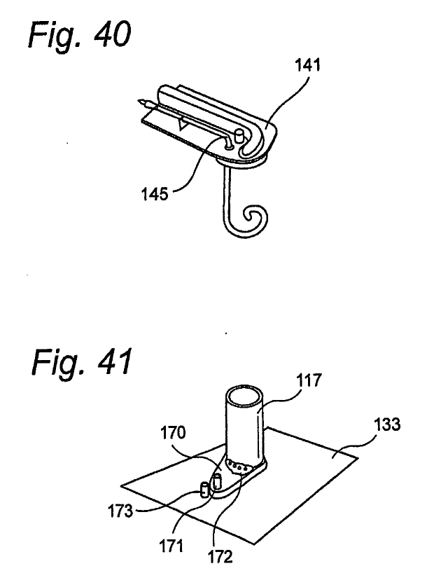

FIG. 40 shows the main cap element 141, which furnishes structures that carry air and simultaneously serves to support a heating bar 145. The main cap 141 and the heating bar 145 are embodied such that when the basic unit 101 is coupled to the main cap 141, both the air-carrying conduits and the electrical connection element 147 of the heating bar 145 are suitably coupled functionally to the basic unit 101.

FIG. 41 illustrates a concept for picking up pressure levels that are indicative of the flow of breathing gas. By this concept, a pickup of a pressure prevailing in the connection stub 117 is made via measuring caverns 172, which are distributed in the circumferential direction of the connection stub 117. The measuring caverns 172 communicate with a first differential pressure measuring conduit 171 via a connection structure 170. A further pressure value can be picked up from the outlet portion 133 via a second differential pressure measuring conduit 173.

FIG. 42 illustrates an equipment concept in which the two modules 104, 103 are closed off via a bottom plate 195, and a bottom seal 196. The bottom plate 195 and bottom seal 196 simultaneously define elastomer portions for the equipment to stand on, allowing the thus-formed basic unit 101 to be set up in a slip-proof, vibration-insulated fashion.

The constructed characteristics described above, particularly the details described in conjunction with FIGS. 16, 18 through 25, 27a, 28 through 30, and in particular each of FIGS. 34 through 42, can also be considered on their own as independent inventive concepts.

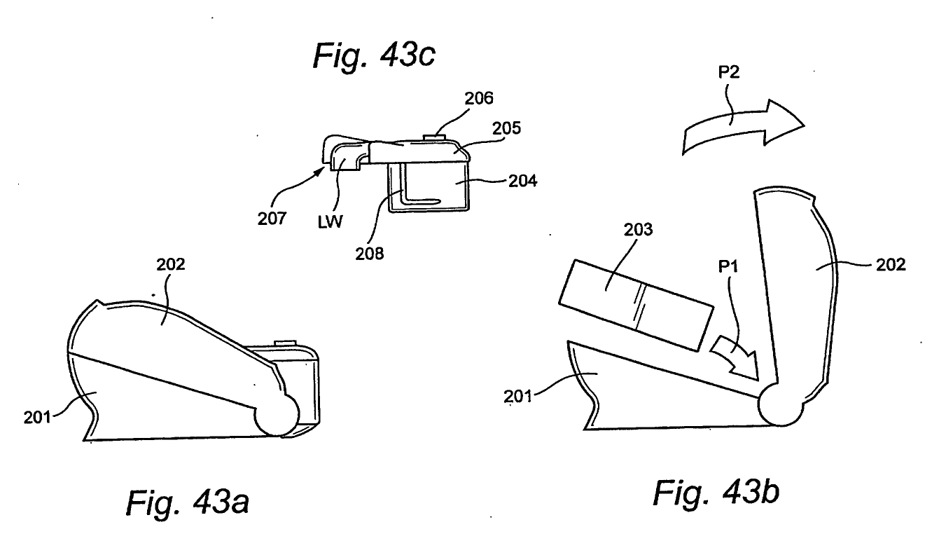

The device shown in FIG. 43a serves to administer a breathing gas at a pressure level that is above the ambient pressure. This device includes a second module for receiving an air delivery device that is not further visible in this view. This air delivery device forms part of a first module, which is inserted into the second module that can be seen here. The second module forms a basic unit, which is constructed divided into a lower structural part 201 and an upper structural part 202. The two structural parts 201, 202 can be put in an open position, as FIG. 43b shows, in which the first module 203 is removable from the basic unit or, as indicated by the arrow symbol P1, is insertable into the basic unit. For attaining this open position, the second housing part 202 is pivoted upward from the lower structural part 201 as indicated by the arrow symbol P2. After the first module 203 has been inserted into the basic unit, the upper structural part can be pivoted back onto the lower structural part counter to the direction indicated by the arrow symbol P2.

The air humidifier shown in FIG. 43c can then be placed on the thus-closed housing unit. The air humidifier shown in FIG. 43c includes a humidifying water container 204 and a bridge part 205, which furnishes an airway LW. The airway LW, in the coupled position, connects a compression-side outlet portion of the first module 203 with an outlet stub portion 206 that leads out of the humidifying water container 204. A flexible breathing gas line can be connected to the outlet stub portion 206 in a manner known per se, and this breathing gas line per se connects the air humidifier to a breathing mask to be applied to the patient. It is possible to embody the bridge part 205 such that it has connection devices 207, through which a heater 208, which dips into the humidifying water container 204, is supplied with electrical energy. It is also possible to accomplish the heating up of the humidifying water, received in the humidifying water container 204, by other kinds of heating concepts, especially with a heating area device that comes into contact with the bottom or wall face of the container 204. The first module 203 can be braced inside the basic unit largely insulated from vibration via elastic contact elements.

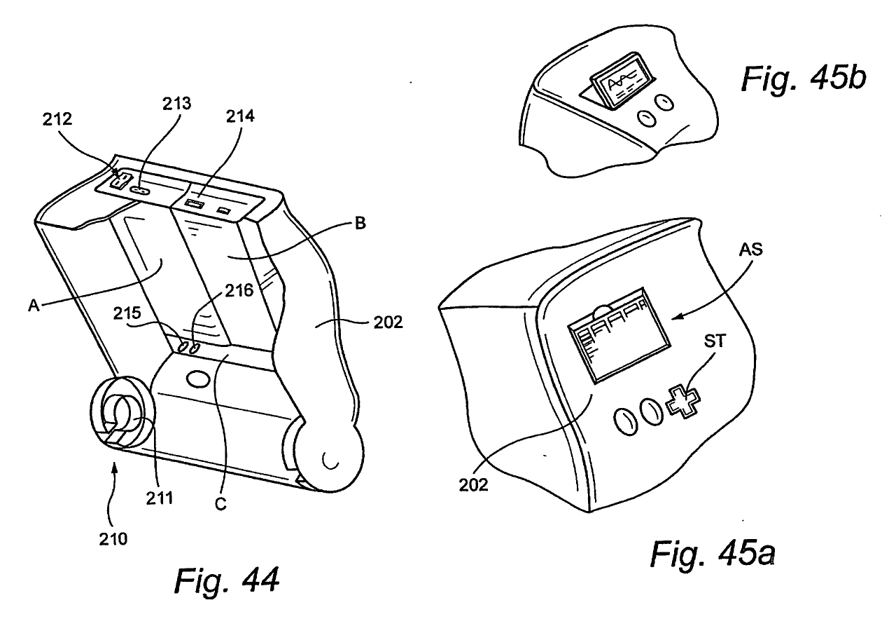

In FIG. 44, the upper structural part 202 is shown partly cutaway. This structural part 202 includes a pivot bearing device 210, which here includes a pivot bearing portion 211 that can be coupled pivotably to a pivot pin, embodied on a lower structural part 201 not further shown here. The pivot bearing device 210 is embodied such that in a predetermined pivoted position, the upper structural part 202 can be removed from the first or lower structural part 201, not further shown here. Two electrical modules A, B are detachably inserted into the upper structural part 202. The electrical module A shown here is a voltage supply circuit, which as such includes a power pack and a power switch 212 as well as a power line connection plug 213. The power switch 212 and the power line connection plug 213 are open to the environment, via a recessed portion embodied in the rear region of the upper structural part 202, and are thus in this sense accessible from outside. The second electrical module B shown here is a module that receives a control circuit. This control circuit includes a plurality of user control areas and display elements, not visible in this view but located in the open toward the top of the upper structural part 202. This module also includes interface devices, embodied in a way only suggested here as a USB interface 214.

It is possible for these two modules A, B to be embodied such that they can be coupled electrically directly to one another in the way required. It is also possible, in the region of the upper structural part 202, to provide a common rail electrical rail C, by way of which a suitable electrical coupling of the electrical components received in the device of the invention can be attained. This electric rail C is embodied in this exemplary embodiment such that the electrical modules A, B can be docked to it. This rail system C also furnishes contacts 215, 216, by way of which an electrical connection with the first module, not visible in this view, is attainable as soon as the upper structural part 202 has been pivoted into the closing position.

In FIG. 45a, it is shown in merely sketchlike form how a recessed portion AS, embodied for instance in the upper structural part 202, might look. In this exemplary embodiment, the recessed portion AS is embodied such that by way of it, the applicable user control areas and display portions embodied on the electrical module B are accessible. A control button ST may be provided, by way of which navigation inside a control window, designed as a screen menu, is possible.

The recessed portion AS may be embodied such that, as sketched in FIG. 45b, a flip display embodied on the electrical module B can be exposed and suitably erected.

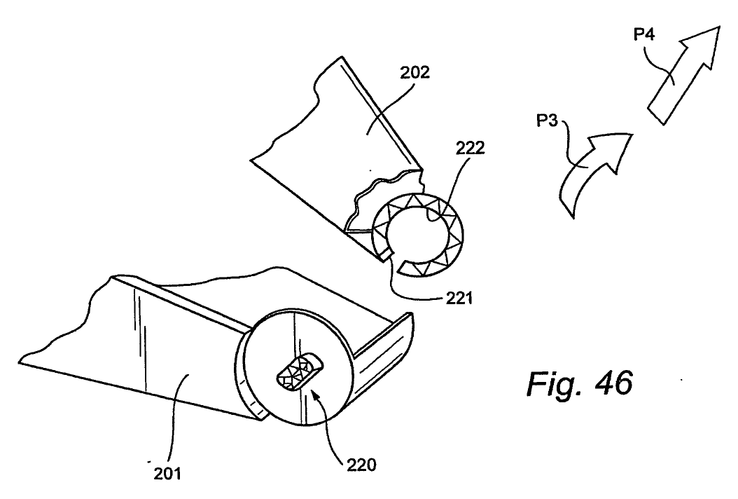

FIG. 46 shows purely as an example how a pivot coupling device, serving the purpose of pivotable and detachable coupling of the lower structural part 201 and upper structural part 202, might be embodied. This pivot coupling device here includes a pivot pin element 220, embodied on the lower structural part 201, which is flattened on diametrically opposed side portions. This pivot pin element 220, via its flattened portions, can pass through an insertion slit 221 into the interior of a pivot bush portion 222 embodied on the upper structural part 202. The thus-embodied pivot coupling device makes it possible first to pivot the upper structural part 202 on the lower structural part 201 (arrow symbol P3) and then, in accordance with the orientation of the flat faces of the pivot pin 220, to remove it to the front or to the top, as indicated by the arrow symbol P4.

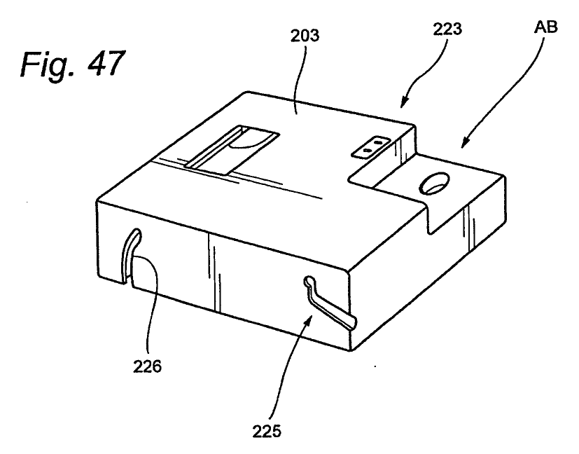

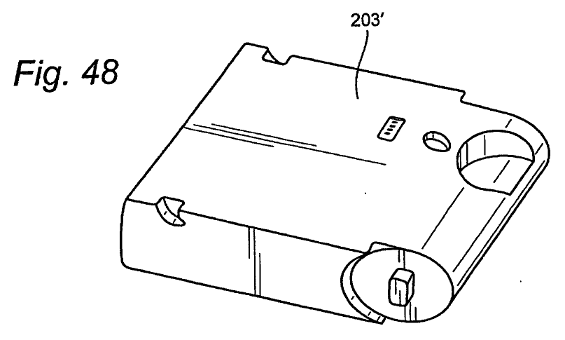

The first module, intended for insertion into the basic unit embodied according to the invention, is shown as an example in FIG. 47. This first module 203 includes an electrical connection portion 223 for creating an electrical connection with the electrical modules A, B (for which see FIG. 44) and the contact elements 215, 216.

The first module 203 forms a compression-side connection portion AB, which in this exemplary embodiment is embodied such that a stub portion, embodied on the bridge element of an air humidifier (for which see FIG. 43c) is insertable directly into it.

Guide structures 225, 226 are embodied on the first module 203, which facilitate the positionally correct insertion and fixation of the first module 203 in the lower structural part 201 (see FIG. 43b). A blower is accommodated in the first module 203, and the power supply of this blower is effected via the electrical connection portions 223 shown in suggested form here. Also located in the first module is one intake-side and one compression-side sound-absorbing section, which is formed by airway portions whose wall comprises a sound-absorbing material. The first module 203 can be embodied as a disposable or replaceable unit, which as such can be replaced like a toner cartridge after the housing device of the invention is opened. It is possible to provide various first modules that are compatible with the basic device of the invention; these first modules may have properties adapted to the particular intended purpose. In particular, for a quick examination, it is possible to use a first module 203 that includes relatively inexpensive electrical components. It is also possible to create an first module 203 that is intended for long-term use and as such has components correspondingly suited for that application.

The device of the invention is preferably dimensioned such that its base has dimensions in the range of approximately 20×20 cm. The height of the device is preferably in the range of approximately 11 cm. The air humidifier module may protrude past the aforementioned base, in particular toward the front.

In FIG. 48, an first module 203′ is shown, which is embodied such that an upper housing module that carries the electrical components can be placed on it, and in particular pivoted onto it. In this design of the first module 203′, it becomes possible optionally to dispense with the lower structural part 201 provided in the second module described above. Otherwise, the above descriptions logically apply, in particular those pertaining to the upper structural part 202.

By the modular concept of the invention, it becomes possible to create a CPAP device which can be broken down into its main components without tools, optionally even by the user himself. Because it is easy to break down, the hygiene-relevant device components, and in particular the first module 203, can be replaced easily. It moreover becomes possible, from the individual compatible modules, to create a device adapted individually to the requirements of the user. The upper structural part 202, as the structural part that predominantly determines the overall appearance of the device of the invention, can be chosen to suit a patient's wishes in terms of color. Preferably, except for the electrical devices of the first module, all the other electrical components are located in the region of the upper structural part 202, or of the individual modules received in it.

The air humidifier device may also be embodied in a different way from the variants described above. In particular, it is possible to embody the air humidifier device such that it is not slipped onto the upper structural part 202 from above but instead can be slid from the front into the upper structural part 202.

It is also possible to embody the basic unit such that the first module is insertable into the interior of the basic unit through an insert opening provided in the rear region of the basic unit or in its side region. In general, the first module can be accommodated in insert-like fashion, replaceably, in a modular structure that is easy to open.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability with patients and non-patients alike in non-medical applications.

1. An apparatus for administering a breathable gas, in particular at a pressure level that at least in some phases is above the ambient pressure, having:
a delivery device for delivering the breathing gas via a breathing gas delivery path,
a breathing gas inlet line region, located upstream of the delivery device and forming part of the breathing gas delivery path,
an outlet line region, downstream of the delivery device and likewise forming part of the breathing gas delivery path, wherein the breathing gas inlet line region and the breathing gas outlet line region are furnished by a first module having at least one sound-absorbing structure.

2. The apparatus as defined by claim 1, wherein the first module housing is embodied as a thin-walled structure.

3. The apparatus as defined by claim 2, wherein the first module housing is made from a foil- or film-coated cardboard material.

4. The apparatus as defined by claim 1, wherein the first module housing is reinforced using a structure enclosed in the first module housing, in particular where the structure comprises one of said at least one sound-absorbing structure.

5. The apparatus as defined by claim 1, wherein the first module housing is adapted to embody the breathing gas duct portions.

6. The apparatus as defined by claim 1, wherein the breathing gas duct portions can be exposed by removing cover portions.

7. The apparatus as defined by claim 1, wherein the cover portions can be detached from the first module housing via perforated places.

8. The apparatus as defined by claim 1, wherein the first module housing includes a connection portion, for connecting a suction- and/or compression-side conduit portion of the delivery device.

9. The apparatus as defined by claim 8, wherein the connection portion is a recess adapted for receiving an insert that includes the delivery device.

10. The apparatus as defined by claim 1, wherein the delivery device is initially integrated with the first module and upon replacement of the first module is removable from the first module.

11. A first module for an apparatus as defined by claim 1.

12. The first module as defined by claim 11, having a substantially rectangular-blocklike structure.

13. The first module as defined by claim 11, wherein the outlet orifice region is adapted for attachment of extension structures for extending a breathing gas delivery conduit.

14. The first module as defined by claim 11, adapted to pick up a signal indicative of the breathing gas flow.

15. A second module for docking a first module as defined by claim 11.

16. The second module as defined by claim 15, further including a contact device to electrically couple with a blower motor located in the first module.

17. The second module as defined by claim 15, further including a blower device.

18. The second module as defined by claim 15, further including a drive device, for driving an impeller provided in the first module.

19. The second module as defined by claim 15, further including a coil device, for exerting a torque on an impeller provided in the region of the first module.

20. The second module as defined by claim 15, further including a drive device and an impeller device, releasably coupled to the drive device, and wherein, using the first module, a connection structure is furnished, for forming an impeller housing that surrounds the impeller device.

21. A structural connection part for furnishing a breathing gas conduit portion, extending downstream of the first module, attached to the first module via a sliding sealing face.

22. A structural connection part for furnishing a breathing gas conduit portion, extending downstream of the first module of claim 11, including a signal pickup device, for pickup of a signal that is indicative of the breathing gas flow.

23. A structural connection part for furnishing a breathing gas conduit portion, extending downstream of the first module of claim 11, that forms part of an air humidifier device.

24. An air humidifier device for a CPAP device, having a structural part that forms a breathing gas conduit, and a signal pickup device for pickup of a signal indicative of the breathing gas flow, in particular a differential pressure signal.

25. An air humidifier device for a CPAP device, having a structural part that forms a breathing gas conduit, and a heating bar device, which is detachably coupled to said structural part.

26. An apparatus for administering a breathable gas, in particular at a pressure level that at least in some phases is above the ambient pressure, having:
a delivery device for delivering the breathing gas via a breathing gas delivery path,
a breathing gas inlet region, located upstream of the delivery device and forming part of the breathing gas delivery path,
an outlet region, downstream of the delivery device and likewise forming part of the breathing gas delivery path, and
a housing structure,
wherein the apparatus in its construction is broken down into a first module and a second module, and the first module includes the delivery path components that serve to furnish the breathing gas delivery path, and the second module includes the apparatus components that can be separated from the delivery path components.

27. The apparatus as defined by claim 26, wherein the first module includes the breathing gas line portion that defines the breathing gas inlet region.

28. The apparatus as defined by claim 26, wherein the breathing gas line portion that defines the breathing gas inlet region is provided with a sound damping device.

29. The apparatus as defined by claim 26, wherein the first module includes a breathing gas line portion that defines the breathing gas outlet region.

30. The apparatus as defined by claim 26, wherein the breathing gas line portion that defines the breathing gas inlet region is provided with a sound damping device.

31. The apparatus as defined by claim 26, wherein the delivery device is integrated, as a replaceable delivery unit, within the first module.

32. The apparatus as defined claim 26, wherein the sound damping device provided in the region of the breathing gas inlet region is integrated, as a replaceable inlet sound damper unit, with the first module.

33. The apparatus as defined by claim 26, wherein the sound damping device provided in the region of the breathing gas outlet region is integrated, as a replaceable outlet sound damper unit, within the first module.

34. The apparatus as defined by claim 26, wherein the two sound damper units are combined into an interchangeable unit.

35. The apparatus as defined by claim 26, wherein the first module has a main body, embodied as a receiving bell, in which the sound damper units and the delivery unit are received.

36. The apparatus as defined by claim 26, wherein the first module includes a pickup device, for picking up a measurement signal that is indicative of the flow of breathing gas.

37. The apparatus as defined by claim 26, wherein the pickup device is embodied as a differential pressure pickup device.

38. The apparatus as defined by claim 26, wherein, using the main body, connection devices are furnished for coupling further elements of the region of the breathing gas delivery path located downstream of the delivery device.

39. The apparatus as defined by claim 26, wherein the connection devices of the first module are embodied and disposed such that they are accessible via a connection portion that is bounded by the second module.

40. The apparatus as defined by claim 26, wherein the first module and the second module are embodied as components substantially stacked one above the other.

41. The apparatus as defined by claim 26, wherein the second module includes an upper housing device, and the equipment operating devices are integrated within the second module.

42. The apparatus as defined by claim 26, wherein the second module includes a mains voltage connection structure.

43. The apparatus as defined by claim 26, wherein the mains voltage connection structure includes a mains switch, provided for manual actuation, and that this mains switch is disposed in the immediate vicinity of where a power cord enters the second module.

44. The apparatus as defined by claim 26, wherein the connection of the power cord to the second module is made via an outlet, and that the mains switch is combined with the outlet to make a structural unit.

45. The apparatus as defined by claim 26, wherein the second module is coupled with a power pack.

46. The apparatus as defined by claim 26, wherein the second module includes a lower structural part intended for receiving the first module and a upper housing part closing off this lower structural part, forming an upper equipment face.

47. The apparatus as defined by claim 26, wherein the equipment furnishes a connection device for selective connection of an air humidifier.

48. The apparatus as defined by claim 26, wherein the air humidifier includes a cup portion and a main cap closing off the cup portion.

49. The apparatus as defined by claim 26, wherein the main cap defines an air supply path, for creating a connecting path between the outlet portion of the first module and the inner region of the cup portion.

50. The apparatus as defined by claim 26, wherein the main cap includes a connection stub for connecting a breathing gas line.

51. The apparatus as defined by claim 26, wherein the connection stub is embodied integrally with the main cap.

52. The apparatus as defined by claim 26, wherein the air humidifier is provided with a heater.

53. The apparatus as defined by claim 26, wherein the heater is formed by a heating bar.

54. The apparatus as defined by claim 26, wherein the heating bar is incorporated detachably into the air humidifier.

55. The apparatus as defined by claim 26, wherein the heating bar is coupled with the main cap.

56. The apparatus as defined by claim 26, wherein the heating bar is guided all the way through the main cap in a sealing manner.

57. The apparatus as defined by claim 26, wherein the heating bar is detachably locked to the main cap.

58. The apparatus as defined by claim 26, wherein the heating bar is provided with an electrical connection element.

59. The apparatus as defined by claim 26, wherein the heating bar is incorporated into the main cap in such a way that the electrical connection element of the heating bar, in the docked state, is coupled with electrical connection device that are provided in the basic unit.

60. The apparatus as defined by claim 26, wherein the main cap includes a partition element, for disconnecting an air path, extending in the main cap, from the inner region of the cup portion in some portions.

61. An apparatus for administering a breathable gas, in particular at a pressure level that at least in some phases is above the ambient pressure, having a delivery device for delivering the breathing gas via a breathing gas delivery path, wherein the apparatus includes a recording device, for recording information indicative of the hygienic condition of the apparatus.

62. The apparatus as defined by claim 61, wherein the recording device is embodied as a memory chip.

63. The apparatus as defined by claim 62, wherein the memory chip is integrated with a blower motor plug.

64. An apparatus for administering a breathable gas, in particular at a pressure level that at least in some phases is above the ambient pressure, having a delivery device, within a basic unit, for delivering the breathing gas via a breathing gas delivery path and an air humidifier device, wherein the air humidifier device includes a container device for holding humidifying water and includes a heater, for heating at least a portion of the humidifying water, and the heater in cooperation with a main cap penetrates into the container device.

65. The apparatus as defined by claim 64, wherein the heater is embodied as a heating element.

66. The apparatus as defined by claim 65, wherein the heating element is detachably coupled to the main cap.

67. The apparatus as defined by claim 64, wherein the heater is embodied such that an electric coupling of the heater to the basic unit is accomplished in the course of the placement of the main cap structure on the basic unit.

68. The apparatus as defined by claim 67, wherein the heater can be realized by an angled heater bar, which has a connection end portion that includes an electrical connection element for supplying electrical power.

69. A device for delivering a breathing gas, having a pumping device for pumping the breathing gas to a pressure level that is above the ambient pressure, and having a housing device for receiving the pumping device, wherein the pumping device forms part of the air feeding module, and the housing device forms a housing system, which is divided up into a lower housing part and an upper housing part, and the two housing parts can be put into an open position toward one another, and in this open position the air feeding module can be removed from or introduced into the housing system.

70. The device in accordance with claim 69, wherein the upper housing part is coupled pivotably to the lower housing part.

71. The device in accordance with claim 69, wherein the upper housing part and the lower housing part each form one housing half-shell.

72. The device in accordance with claim 69, wherein the two housing parts are detachably coupled to one another.

73. The device in accordance with claim 69, wherein the two housing parts are coupled to one another via a joint structure, and the joint structure is embodied such that a pivot axis extends in the region of a front or rear crosswise edge of the housing device.

74. The device in accordance with claim 69, wherein the air feeding module is embodied as a boxlike insert.

75. The device in accordance with claim 69, wherein a control circuit and a voltage supply circuit are inserted detachably into the upper housing part.

76. The device in accordance with claim 69, wherein the control circuit and the voltage supply circuit are each divided into separate electrical modules.

77. The device in accordance with claim 69, wherein an air humidifier device is provided, which can be incorporated into the device in such a way that it communicates with an airway portion that is furnished by the air feeding module.
